(19) United States
(12) Patent Application Publication
(10) Pub. No.: US 2025/0288416 A1
(43) Pub. Date: Sep. 18, 2025

(54) ANCHOR HOOK FOR SIDE-DELIVERY TRANSCATHETER HEART VALVE PROSTHESIS

(71) Applicant: VDyne, Inc., Maple Grove, MN (US)

(72) Inventors: Mark CHRISTIANSON, Plymouth, MN (US); Robert VIDLUND, Forest Lake, MN (US); Chad PERRIN, Andover, MN (US); Craig EKVALL, East Bethel, MN (US)

(73) Assignee: VDyne, Inc., Maple Grove, MN (US)

(21) Appl. No.: 19/223,965

(22) Filed: May 30, 2025

Related U.S. Application Data
(63) Continuation of application No. 17/372,022, filed on Jul. 9, 2021, now Pat. No. 12,343,256.
Application No. 17/372,022 is a continuation of application No. PCT/US2020/013240, filed on Jan. 11, 2020.
(60) Provisional application No. 62/790,465, filed on Jan. 10, 2019.
(60) Provisional application No. 62/847,741, filed on May 14, 2019.

(51) Int. Cl. A61F 2/24; A61B 17/00; A61B 17/34; A61F 2/91

(52) U.S. Cl. CPC A61F 2/2436; A61B 17/3468; A61F 2/2418; A61B 2017/00243; A61B 2017/00292; A61B 2017/00358; A61F 2/91; A61F 2210/0014; A61F 2220/0016; A61F 2230/0017; A61F 2250/0098

(57) ABSTRACT

The invention relates to an anchor hook and methods of using the same for subannular anchoring of a transcatheter heart valve replacement, and in particular for an orthogonally delivered (side-delivered) transcatheter prosthetic heart valve having a annular support frame having compressible wire cells that facilitate rolling and folding the valve length-wise, or orthogonally to the central axis of the flow control component, allowing a very large diameter valve to be delivered and deployed to the mitral or tricuspid valve from the inferior vena cava or superior vena cava, or trans-septally to the mitral valve, the valve having a height of about 5-60 mm and a diameter of about 25-80 mm, without requiring an oversized diameter catheter and without requiring a delivery catheter to bend 90 degrees during deployment.

### CROSS-REFERENCE TO RELATED APPLICATIONS

This application is a continuation of U.S. patent application Ser. No. 17/372,022, filed Jul. 9, 2021, entitled “Anchor Hook for Side-Delivery Transcatheter Heart Valve Prosthesis,” which is a continuation of International Patent Application No. PCT/US2020/013240, filed Jan. 11, 2020, entitled “Anchor Hook for Side-Delivery Transcatheter Heart Valve Prosthesis,” which claims priority to and the benefit of U.S. Provisional Patent Application No. 62/847,741, filed May 14, 2019, entitled “Retrievable Anchor Hook for Side-Delivery Transcatheter Heart Valve Prosthesis” and U.S. Provisional Patent Application No. 62/790,465, filed Jan. 10, 2019, entitled “Subannular Securement Anchors for Orthogonal Transcatheter Heart Valve Prosthesis,” the disclosure of each of which is incorporated herein by reference in its entirety.

### BACKGROUND OF THE INVENTION

### Field of the Invention

The invention relates to an anchor hook and methods of using the same for subannular anchoring of a transcatheter heart valve replacement (A61F2/2412).

### Description of the Related Art

Embodiments are described herein that relate to prosthetic heart valves, and devices and methods for use in the delivery and deployment of such valves.

Prosthetic heart valves can pose challenges for delivery and deployment within a heart, particularly for delivery by catheters through the patient's vasculature rather than through a surgical approach.

Delivery of traditional transcatheter prosthetic valves generally includes compressing the valve in a radial direction and loading the valve into a delivery catheter such that a central annular axis of the valve is parallel to the lengthwise axis of the delivery catheter. The valves are deployed from the end of the delivery catheter and expanded outwardly in a radial direction from the central annular axis. The expanded size (e.g., diameter) of traditional valves, however, can be limited by the internal diameter of the delivery catheter. The competing interest of minimizing delivery catheter size presents challenges to increasing the expanded diameter of traditional valves (e.g., trying to compress too much material and structure into too little space).

Accordingly, a need exists for prosthetic valves with one or more anchoring features while maintaining a relatively small compressed size that allows for transcatheter delivery of the valve.

### BRIEF SUMMARY OF THE INVENTION

The present invention is directed to an anchor hook system for a transcatheter heart valve replacement.

Accordingly, the present invention is directed to an anchoring system for a transcatheter prosthetic tricuspid or mitral heart valve, comprising:

an anchor hook having an elongated shaft portion and a hook portion at a distal end of the elongated shaft portion, a shaft tether loop on the shaft portion, and a tether mount on the hook portion,

a plurality of anchoring tabs or barbs disposed along the shaft portion,

a delivery catheter,

an anchor channel disposed within the valve,

an elongated pusher cable detachably attached to a proximal end of the shaft portion, and extending proximally through the delivery catheter,

an elongated tether attached to the tether mount and threaded through the shaft loop, and extending proximally through the delivery catheter,

the anchor hook made from shape memory material and the anchor hook heat-set into an open or expanded configuration where the hook portion extends away from a central axis of the shaft portion, and the anchor hook adjustable to a closed or compressed configuration by exerting a pulling force on the tether in a proximal direction where the hook portion is folds towards the shaft portion,

the anchoring tabs or barbs heat-set into an open or expanded configuration where the tabs or barbs extend away from the central axis of the shaft portion, and the tabs or barbs adjustable to a closed or compressed configuration by exerting a pulling force on the pusher cable in a proximal direction where the tabs or barbs are folded towards the shaft portion as the shaft portion is drawn into the delivery catheter, where the anchoring tabs or barbs in an expanded configuration have a radius from the central axis larger than an inner diameter of the anchor channel.

In another preferred embodiment, the anchoring system includes wherein the channel is integrated into or attached to an exterior surface of a valve body portion of the transcatheter prosthetic tricuspid or mitral heart valve.

In another preferred embodiment, the anchoring system includes wherein the channel is integrated into or attached to an interior surface of a valve body portion of the transcatheter prosthetic tricuspid or mitral heart valve.

In another preferred embodiment, the anchoring system includes wherein the shaft portion is selected from a single elongated member with the anchoring tabs or barbs extending away from the elongated member, or a chain of diamond-shaped cells with the anchoring tabs or barbs extending from lateral points of the diamond-shaped cells.

In another preferred embodiment, the anchoring system includes wherein the hook portion is selected from a single diamond-shaped cell, or a diamond-shaped cell with one or more anchoring tabs or barbs extending from lateral points of the diamond-shaped cell.

In another preferred embodiment, the anchoring system includes a second anchor hook attached to a second pusher cable and a second tether, said second cable and tether disposed within a second delivery catheter, said second catheter configured to be inserted thru a second anchor channel.

In another preferred embodiment, the anchoring system includes wherein the anchor hook is compressed within a cylindrical channel integrated into or attached to a valve body portion of the transcatheter prosthetic tricuspid or mitral heart valve, and wherein the cylindrical channel extends through the valve body vertically or at an angle up to 45 degrees from vertical, wherein vertical is parallel to a central atrial-to-ventricle axis of the transcatheter prosthetic tricuspid or mitral heart valve.

In another preferred embodiment, the anchoring system includes wherein the shaft has between 2-6 tabs or barbs, and wherein the channel is a cylinder of polyester or is cylinder lined with polyester.

In yet another preferred embodiment, the invention includes an orthogonally delivered (side-delivered) transcatheter prosthetic heart valve having an anchoring system, comprising:

the anchoring system;

a self-expanding annular support frame, the anchoring system mounted on or within the annular support frame, said annular support frame having a central channel and an outer perimeter wall circumscribing a central vertical axis in an expanded configuration, said annular support frame having a distal side and a proximal side,

a flow control component mounted within the annular support frame and configured to permit blood flow in a first direction through an inflow end of the valve and block blood flow in a second direction, opposite the first direction, through an outflow end of the valve,

wherein the valve is compressible to a compressed configuration for introduction into the body using a delivery catheter for implanting at a desired location in the body, said compressed configuration is oriented along a horizontal axis at an intersecting angle of between 45-135 degrees to the central vertical axis, and expandable to an expanded configuration having a horizontal axis at an intersecting angle of between 45-135 degrees to the central vertical axis,

wherein the horizontal axis of the compressed configuration of the valve is substantially parallel to a length-wise cylindrical axis of the delivery catheter,

wherein the valve has a height of about 5-60 mm and a diameter of about 25-80 mm.

In yet another preferred embodiment, the valve includes wherein the channel has a radio-opaque marker.

In yet another preferred embodiment, the valve includes wherein the channel is braided polyethylene, treated pericardial tissue, ePTFE, or Nitinol.

In yet another preferred embodiment, the valve includes wherein the tether or strap has a tooth-portion and a slidable locking element with a tooth-engaging pawl element.

In yet another preferred embodiment, the valve includes wherein the annular support frame further comprises a distal anchoring tab mounted on the distal side of the annular support frame, and a proximal anchoring tab mounted on the proximal side of the annular support frame.

In yet another preferred embodiment, the valve includes wherein the annular support frame is comprised of a plurality of compressible wire cells having an orientation and cell geometry substantially orthogonal to the central vertical axis to minimize wire cell strain when the annular support frame is configured in a vertical compressed configuration, a rolled compressed configuration, or a folded compressed configuration.

In yet another preferred embodiment, the valve includes wherein the annular support frame has a lower body portion and an upper collar portion, wherein the lower body portion in an expanded configuration forms a shape selected from a funnel, cylinder, flat cone, or circular hyperboloid.

In yet another preferred embodiment, the valve includes wherein said annular support frame is comprised of a braided, wire, or laser-cut wire frame, and said annular support frame is covered with a biocompatible material.

In yet another preferred embodiment, the valve includes wherein the annular support frame has a side profile of a flat cone shape having a diameter R of 40-80 mm, a diameter r of 20-60 mm, and a height of 5-60 mm.

In yet another preferred embodiment, the valve includes wherein the annular support frame has an inner surface and an outer surface, said inner surface and said outer surface covered with a biocompatible material selected from the following consisting of: the inner surface covered with pericardial tissue, the outer surface covered with a woven synthetic polyester material, and both the inner surface covered with pericardial tissue and the outer surface covered with a woven synthetic polyester material.

In yet another preferred embodiment, the valve includes wherein the annular support frame has a side profile of an hourglass shape having a top diameter R1 of 40-80 mm, a bottom diameter R2 of 50-70 mm, an internal diameter r of 20-60 mm, and a height of 5-60 mm.

In yet another preferred embodiment, the valve includes wherein the valve in an expanded configuration has a central vertical axis that is substantially parallel to the first direction.

In yet another preferred embodiment, the valve includes wherein the flow control component has an internal diameter of 20-60 mm and a height of 10-40 mm, and a plurality of leaflets of pericardial material joined to form a rounded cylinder at an inflow end and having a flat closable aperture at an outflow end.

In yet another preferred embodiment, the valve includes wherein the flow control component is supported with one or more longitudinal supports integrated into or mounted upon the flow control component, the one or more longitudinal supports selected from rigid or semi-rigid posts, rigid or semi-rigid ribs, rigid or semi-rigid battens, rigid or semi-rigid panels, and combinations thereof.

In yet another preferred embodiment, the valve includes wherein the distal anchoring tab is comprised of wire loop, a wire frame, a laser cut frame, an integrated frame section, or a stent, and the distal anchoring tab extends from about 10-40 mm away from the distal side of the annular support frame.

In yet another preferred embodiment, the valve includes wherein the proximal anchoring tab is comprised of wire loop, a wire frame, a laser cut frame, an integrated frame section, or a stent, and the distal anchoring tab extends from about 10-40 mm away from the proximal side of the annular support frame.

In yet another preferred embodiment, the valve includes further comprising an upper distal anchoring tab attached to a distal upper edge of the annular support frame, the upper distal anchoring tab comprised of wire loop, a wire frame, a laser cut frame, an integrated frame section, or a stent, and extends from about 2-20 mm away from the annular support frame.

In yet another preferred embodiment, the valve includes wherein the outer perimeter wall comprises a front wall portion that is a first flat panel and a back wall portion that is a second flat panel, and wherein a proximal fold area and a distal fold area each comprise a sewn seam, a fabric panel, a rigid hinge, or a flexible fabric span without any wire cells.

In yet another preferred embodiment, the valve includes wherein the annular support frame is comprised of compressible wire cells selected from the group consisting of braided-wire cells, laser-cut wire cells, photolithography produced wire cells, 3D printed wire cells, wire cells formed from intermittently connected single strand wires in a wave shape, a zig-zag shape, or spiral shape, and combinations thereof.

In yet another preferred embodiment, the invention includes a method for deploying an anchor hook system for a prosthetic transcatheter heart valve, comprising:
(i) advancing a delivery catheter thru an anchor channel of a transcatheter heart valve to a subannular position,
(ii) pushing an anchor hook out of a distal end of the delivery catheter using an elongated pusher cable that is disposed within the delivery catheter and attached to a proximal end of a shaft portion of the anchor hook, the shaft portion having anchoring tabs or barbs disposed along the shaft portion and a hook portion attached to a distal end of the shaft portion,

wherein the hook portion and the anchoring tabs or barbs are initially compressed against the shaft portion when within the delivery catheter, and the hook portion and the anchoring tabs or barbs expand to a heat-set shape-memory open configuration when expelled from the delivery catheter,
(iii) pulling the shaft portion of the anchor hook into the anchor channel by pulling the pusher cable in a proximal direction,

wherein pulling the shaft portion retracts the hook portion in a proximal direction and captures native leaflet tissue or native chordae tendineae within or on the hook portion of the anchor hook, and the retracted hook portion presses the native leaflet tissue or native chordae tendinea against a subannular portion of the prosthetic transcatheter heart valve or a subannular surface of the native annulus, and

wherein pulling the shaft portion of the anchor hook into the anchor channel draws the opened/expanded tabs/barbs into the anchor channel, where the anchor channel has a narrower cylindrical radius than the extended radius of the tabs/barbs and causes the tabs/barbs to partially fold toward the shaft portion, and create a tensioning force with the tabs/barbs pressing against an interior surface of the anchor channel, wherein the anchor channel is a mesh or braid and the tabs/barbs penetrate or grip the interior surface of the anchor channel.

In yet another preferred embodiment, the method of deploying includes wherein the native leaflet tissue is tricuspid leaflet tissue.

In yet another preferred embodiment, the method of deploying includes wherein the native leaflet tissue is mitral leaflet tissue.

In yet another preferred embodiment, the method of deploying includes wherein the native leaflet tissue is mitral leaflet tissue, and wherein the anchor hook system comprises, a first anchor hook with hook portion extended and then retracted to capture a P2 mitral leaflet, and a second anchor hook with hook portion extended and then retracted to capture a A2 mitral leaflet.

In yet another preferred embodiment, the method of deploying includes the step of retrieving the anchor hook, wherein the delivery catheter slides over the shaft portion while the tabs/barbs are engaging the anchor channel, and flattens the tabs/barbs back towards or against the shaft portion, thus disengaging the tabs/barbs from the anchor channel, wherein the delivery catheter is advanced distally to a subannular/ventricular position to release the hook portion from the captured native tissue, wherein a tether that is attached to the anchor hook is pulled and folds the hook portion against the shaft portion, wherein the entire anchor hook is then pulled into the delivery catheter, and wherein the delivery catheter is withdrawn and/or the anchor hook is redeployed.

In yet another preferred embodiment, the invention includes a process for manufacturing an anchoring hook for an orthogonally delivered (side-delivered) transcatheter prosthetic heart valve frame, comprising: using additive or subtractive metal or metal-alloy manufacturing to produce the anchoring hook, wherein the additive metal or metal-alloy manufacturing is 3D printing or direct metal laser sintering (powder melt), and wherein the subtractive metal or metal-alloy manufacturing is photolithography, laser sintering/cutting, CNC machining, electrical discharge machining.

In yet another preferred embodiment, the invention includes a method of using the anchoring hook system for orthogonal delivery of implantable prosthetic heart valve to a desired location in the body, the method comprising the steps:
(i) advancing a delivery catheter to the desired location in the body and delivering an expandable prosthetic heart valve with an anchoring hook system to the desired location in the body by releasing the valve from the delivery catheter, and
(ii) anchoring an anchoring hook attached to the valve into native tissue,

wherein the valve comprises an annular support frame having a flow control component mounted within the annular support frame and configured to permit blood flow in a first direction through an inflow end of the valve and block blood flow in a second direction, opposite the first direction, through an outflow end of the valve, the anchoring hook system attached to the annular support frame, a distal anchoring tab mounted on a distal side of the annular support frame, and a proximal anchoring tab mounted on a proximal side of the annular support frame,

wherein the valve is compressible to a compressed configuration for introduction into the body using a delivery catheter for implanting at a desired location in the body, said compressed configuration having a long-axis oriented at an intersecting angle of between 45-135 degrees to the first direction, and expandable to an expanded configuration having a long-axis oriented at an intersecting angle of between 45-135 degrees to the first direction,

wherein the long-axis of the compressed configuration of the valve is substantially parallel to a length-wise cylindrical axis of the delivery catheter, and

wherein the valve has a height of about 5-60 mm and a diameter of about 25-80 mm.

In yet another preferred embodiment, the method of using includes wherein releasing the valve from the delivery catheter is selected from the steps consisting of:
(i) pulling the valve out of the delivery catheter using a rigid elongated pushing rod/draw wire that is releasably connected to the distal side of the valve, wherein advancing the pushing rod away from the delivery catheter pulls the compressed valve out of the delivery catheter, or
(ii) pushing the valve out of the delivery catheter using a rigid elongated pushing rod that is releasably connected to the proximal side of the valve, wherein advancing the pushing rod out of from the delivery catheter pushes the compressed valve out of the delivery catheter.

In yet another preferred embodiment, the method of using includes wherein the step of anchoring an anchoring hook attached to the valve into native tissue, comprises the steps of expelling the anchoring hook from the channel, extending the hook portion of the anchoring hook, capturing native tissue, and pulling the tether to compress the anchoring hook.

In yet another preferred embodiment, the method of using includes the additional step of positioning the distal anchoring tab of the heart valve prosthesis into the right ventricular outflow tract of the right ventricle.

In yet another preferred embodiment, the method of using includes the additional steps of positioning the distal anchoring tab of the heart valve prosthesis into the right ventricular outflow tract of the right ventricle, and positioning an upper distal anchoring tab into a supra-annular position, and the upper distal anchoring tab providing a supra-annular downward force in the direction of the ventricle and distal anchoring tab providing a sub-annular upward force in the direction of the atrium.

In yet another preferred embodiment, the method of using includes the additional step of rotating the heart valve prosthesis using a steerable catheter along an axis parallel to the plane of the valve annulus.

In yet another preferred embodiment, the invention includes a method for orthogonal delivery of implantable prosthetic heart valve having the anchoring hook system in the body, the method comprising the steps:
(i) advancing a distal end of a guide wire to a distal location, wherein the distal location is a pulmonary artery or a left ventricle of a heart, wherein the guide wire starts outside of a patient using femoral vein access or brachiocephalic vein access, and extends through an inferior vena cava or a superior vena cava to a right atrium, and extends from the right atrium through the tricuspid valve to the pulmonary artery or extends from the right atrium across the atrial septum in a transseptal access through the mitral valve and into a left ventricle;
(ii) advancing a delivery catheter over the guide wire to a target location, where the target location is a right atrium of the tricuspid valve or a left atrium of the mitral valve;
(iii) advancing and delivering an orthogonally compressed (compressed for side-delivery) self-expandable prosthetic heart valve to the target location in the body, wherein a compressed configuration of the valve has a long-axis substantially parallel to a length-wise cylindrical axis of the delivery catheter,

wherein the expanded configuration of the valve has a height of about 5-60 mm and a diameter of about 25-80 mm,

wherein the valve comprises an annular support frame having a flow control component mounted within the annular support frame and configured to permit blood flow in a first direction through an inflow end of the valve and block blood flow in a second direction, opposite the first direction, through an outflow end of the valve,

a distal anchoring tab is mounted on a distal side of the annular support frame, the distal anchoring tab having a length of 10-40 mm and a width of 2-10 mm, wherein the guide wire is threaded through a threading aperture on or within the distal anchoring tab,

at least one proximal anchoring tab is mounted on a proximal side of the annular support frame, the proximal anchoring tab having a length of 2-25 mm and a width of 2-10 mm, and

a valve advancing tool comprising an elongated sheath wherein the guide wire is within a lumen of the sheath, wherein the outer diameter of the sheath is larger than the inner diameter of the threading aperture on the distal anchoring tab, wherein when the sheath is advanced over the guide wire in a distal direction, and a distal end of the sheath contacts a proximal surface of the threading aperture, the valve is advanced distally through the delivery catheter by the distally-directed pulling force that the sheath imparts to the distal anchoring tab;
(iv) partially releasing the valve from the delivery catheter by advancing the sheath over the guide wire, and positioning the distal anchoring tab at a desired anchoring area of the target location,

wherein the desired anchoring area is selected from a right ventricular outflow tract (RVOT) of a right ventricle, and a sub-annular area below an A1-P1 antero-lateral commissure of a mitral valve,

wherein positioning the distal anchoring tab holds the valve at a raised angle of at least 30 degrees to a localized annular plane relative to the horizontal axis of the valve and the delivery catheter,

wherein partially releasing the valve permits blood to flow partially around the prosthetic valve and through the native leaflets, and partially through the flow control component of the prosthetic valve to provide a gradual blood flow transition from flow through native leaflets to complete flow through the prosthetic valve;
(v) completing release of the entire valve from the delivery catheter by advancing the sheath over the guide wire, seating the valve in the native annulus by applying a downward force in the direction of the ventricle;
(vi) seating the at least one proximal anchoring tab at a second desired anchoring area; and
(vii) capturing native tissue using the anchor hook.

In yet another preferred embodiment, the invention includes a method for improving hemodynamic flow during implantation of a transcatheter prosthetic heart valve, comprising:
(i) advancing a delivery catheter to the desired location in the body and delivering the valve of claim 9 to the desired location in the body;
(ii) partially releasing the valve from the delivery catheter to establish blood flow around the partially released valve and establish blood flow through the flow control component;
(iii) completely releasing the valve from the delivery catheter while maintaining attachment to the valve with a positioning catheter or wire to transition to increased blood flow through the flow control component and decreasing blood flow around the valve; and
(iv) deploying the valve into a final mounted position to transition to complete blood flow through the flow control component and minimal or no blood flow around the valve;
(v) anchoring the valve using the anchor hook; and
(vi) disconnecting and withdrawing the positioning catheter or wire from the valve.

In yet another preferred embodiment, the method for improving hemodynamic flow includes wherein the distal anchoring tab is an RVOT tab positioned in the RVOT during the transition from partial release of the valve to complete release of the valve.

### BRIEF DESCRIPTION OF THE SEVERAL VIEWS OF DRAWING

FIG. 1 is an illustration of an anchor hook according to the invention.

FIG. 2 is an illustration of a side view of an anchor delivery system according to the invention.

FIGS. 3-6 are illustrations of a step of a multi-part series showing a side view of a process of using an anchor hook from a delivery catheter.

FIG. 7 is an illustration of a step of a series showing a side view of a process of expelling an anchor hook to a subannular position using a delivery catheter.

FIG. 8 is an illustration of a step of a series showing a side view of a process of capturing native tissue by pulling the tether to re-fold the hook portion against the shaft portion.

FIGS. 9-15 are an illustration of a step of a series showing a side view of a process of capturing native tissue by pulling the tether to re-fold the hook portion against the shaft portion.

FIG. 16 is an illustration of a side view of a heart.

FIG. 17 is an illustration of a top view of a native tricuspid valve with location of inferior vena cava, location of pulmonary artery, and locations of the septal, posterior, and anterior leaflets.

FIG. 18 is an illustration of a side perspective view of a tricuspid leaflets in isolation.

FIG. 19 is an illustration of an orthogonal transcatheter valve replacement (TVR) valve against illustrations of the location of native tissue, with IVC, PA, and the septal, posterior, and anterior leaflets, with an indicator of relative depths A-B-C referring to (A) above the annulus, (B) at or near the annular plane, and (C) below the annulus.

FIG. 20 is an illustration of a side view of a series (left to right) showing how an anchor hook can progress from (i) a stowed position within a delivery catheter sheath/channel, (ii) to an extended anchor position, (iii) to an extended open position, and (iv) to a retracted position, with an indicator of relative depths A-B-C referring to (A) above the annulus, (B) at or near the annular plane, and (C) below the annulus, according to the invention.

FIG. 21 is an illustration of a valve delivery process for an orthogonally delivered (side-delivered) valve, and shows a side view of a heart, with dashed circle showing a tricuspid region of interest.

FIG. 22 is an illustration of a valve delivery process for an orthogonally delivered (side-delivered) valve, and shows a side view of a guide wire step of a multi-step delivery process.

FIG. 23 is an illustration of a valve delivery process for an orthogonally delivered (side-delivered) valve, and shows a side view of a catheter delivery step of a multi-step delivery process, with a catheter containing an orthogonally compressed valve delivered via the IVC.

FIG. 24 is an illustration of a valve delivery process for an orthogonally delivered (side-delivered) valve, and shows a side view of a valve deployment step of a multi-step delivery process, with a catheter expelling an expanded uncompressed valve partially into the tricuspid annulus, with RVOT tab placed in the pulmonary artery right ventricular outflow tract (RVOT) anchoring area.

FIG. 25 is an illustration of a valve delivery process for an orthogonally delivered (side-delivered) valve, and shows a side view of a catheter delivery step of a multi-step delivery process, with a TVR valve fully seated within the tricuspid annulus, RVOT tab, and proximal tab positioned to secure the valve, and subannular anchor devices delivered and inserted into position on or in the valve.

FIG. 26 is an illustration of an anchor hook delivery process, and shows a side view TVR valve fully seated within the tricuspid annulus, RVOT tab, and proximal tab positioned to secure the valve, and subannular anchor devices extended subannularly to capture native tissue, e.g. annular ring, chordae, and/or leaflet.

FIG. 27 is an illustration of an anchor hook delivery process, and shows a side view of a TVR valve fully seated within the tricuspid annulus, RVOT tab, and proximal tab positioned to secure the valve, and subannular anchor devices retracted after capture of native tissue, e.g. annular ring, chordae, and/or leaflet.

FIG. 28 is an illustration of side perspective view of the relation of the anchor hook(s) to native tissue during the anchoring-tissue capture process, with an indicator of relative depths A-B-C referring to (A) above the annulus.

FIG. 29 is an illustration of side perspective view of the relation of the anchor hook(s) to native tissue during the anchoring-tissue capture process, with an indicator of relative depths A-B-C referring to (B) at or near the annular plane.

FIG. 30 is an illustration of side perspective view of the relation of the anchor hook(s) to native tissue during the anchoring-tissue capture process, with an indicator of relative depths A-B-C referring to (C) below the annulus.

FIG. 31 is an illustration of an anchor hook that has been adjusted from a compressed configuration, to an extended/opened configuration to capture native tissue.

FIG. 32 is an illustration of an anchor hook that has been adjusted from an open position to a retracted, and/or cinched configuration.

FIG. 33 is an illustration of a side perspective view of an anchor delivery catheter delivering an anchor hook to a radiomarker spot on the valve collar, according to the invention.

FIG. 34 is an illustration of a side perspective view of an anchor hook deployed to a sub-annular position, according to the invention.

FIG. 35 is an illustration of a side perspective view of an anchor hook that has been deployed to a sub-annular position, and then retracted to capture the chordae and part of the anterior leaflet, according to the invention.

FIG. 36 is an illustration of a side perspective view a transcatheter delivery catheter that has access to the mitral valve from the IVC thru a trans-septal access to deliver an orthogonally deliverable (side-deliverable) mitral valve replacement prosthesis.

FIG. 37 is an illustration of a side perspective view a mitral valve embodiment having the anchor hooks deployed to an expanded position, according to the invention.

FIG. 38 is an illustration of a side perspective view a mitral valve embodiment having the anchor hooks compressed and/or cinched to capture native tissue, according to the invention.

FIG. 39 is an illustration of one embodiment of an anchor hook according to the invention.

FIG. 40 is an illustration of one embodiment of an anchor hook disposed within a delivery catheter that extends through an anchor channel in the valve body to a subannular tissue-capture position, according to the invention.

FIG. 41 is an illustration of one embodiment of an anchor hook released from the delivery catheter allowing the shape-memory material to expand with anchoring tabs or barbs extending laterally, and hook portion extending away from the shaft portion, according to the invention.

FIG. 42 is an illustration of one embodiment of an anchor hook partially pulled back into the anchor channel with anchoring tabs or barbs compressed into the lumen of the anchor channel, and lower shaft portion and hook portion remaining outside of the anchor channel for capture and securement/anchoring of native tissue to the valve body, according to the invention.

FIG. 43 is an illustration of a native mitral valve from a top perspective view above the annular plane from a trans-septal access side view, and shows the A2 leaflet and P2 leaflet in a partially open position.

FIG. 44 is an illustration of a native mitral valve from a bottom perspective view below the annular plane from a P2 side view, and shows the A2 leaflet and P2 leaflet in a partially open position.

FIG. 45 is an illustration of a native mitral valve from a bottom perspective view below the annular plane from an A2 side view, and shows the A2 leaflet and P2 leaflet in a partially open position.

FIG. 46 is an illustration of a prosthetic transcatheter heart valve from a plan or side view, and shows a pair of anchor hooks, each disposed within an anchor channel on the exterior surface of the valve body.

FIG. 47 is an illustration of a prosthetic transcatheter heart valve from a plan or side view, and shows a pair of anchor hooks, each disposed within an anchor channel within the cylinder of the valve body.

FIG. 48 is an illustration of an anchor in a stowed or compressed delivery position.

FIG. 49 is an illustration of an anchor in an expanded deployed position.

FIG. 50 is an illustration of an embodiment of a bare metal anchor element, according to the invention.

FIG. 51 is an illustration of an embodiment of an anchor element having a polyester cover along the shank of the anchor, according to the invention.

FIG. 52 is an illustration of an embodiment of a split shank anchor element having a polyester cover along the split shank of the anchor, according to the invention.

FIG. 53 is an illustration of an embodiment of a split shank anchor element having a bare metal split shank and a polyester cover on the bend or hook portion of the of the anchor, according to the invention.

FIG. 54 is an illustration of an embodiment of a split shank anchor element having a bare metal split shank and a polyester cover on the bend or hook portion of the of the anchor, according to the invention.

FIG. 55 is an illustration of a side perspective view of an orthogonally deliverable transcatheter heart valve with external anchor channel(s) positioned on the exterior of the valve, and extending from cuff/collar along the outer side surface of the valve body to the subannular/lower side, with distal right ventricular outflow tract (RVOT) tab, and proximal tab also shown, according to the invention.

FIG. 56 is an illustration of a side perspective view of a channel having a mesh or tissue channel cover.

FIG. 57 is an illustration of varying diameter sizes of the channel(s).

FIG. 58 is an illustration of varying channel length(s) of the channel(s).

FIG. 59 is an illustration of a side perspective view of an embodiment of a channel and shows braided polyethylene as material for the channel.

FIG. 60 is an illustration of a side perspective view of an embodiment of a channel and shows tissue as material for the channel.

FIG. 61 is an illustration of a side perspective view of an embodiment of a channel and shows expanded polytetrafluoroethylene (ePTFE), as material for the channel.

FIG. 62 is an illustration of a side perspective view of an embodiment of a channel and shows Nitinol® tube or stent, as material for the channel.

FIG. 63 is an illustration of radio-opaque marker locations on a channel, and specifically a top ring location.

FIG. 64 is an illustration of radio-opaque marker locations on a channel, and specifically top and bottom ring locations.

FIG. 65 is an illustration of radio-opaque marker locations on a channel, and specifically top and bottom alignment dot locations.

FIG. 66 is an illustration of radio-opaque marker locations on a channel, and specifically a side strip(s) location.

FIG. 67 is an illustration of radio-opaque marker locations on a channel, and specifically a mid-band location.

FIG. 68 is an illustration of a side view of a channel-less anchor deployment system with a tunneling catheter having a guide rod, a compressed tether lock, a compressed subannular anchor, disposed in a compressed configuration within the catheter, according to the invention.

FIG. 69 is an illustration of a side view of a channel-less anchor deployment system with a tunneling catheter having a guide rod, a compressed tether lock, a partially expelled, partially uncompressed subannular anchor, according to the invention.

FIG. 70 is an illustration of a side view of a channel-less anchor deployment system with a tunneling catheter having an uncompressed expanded tether lock, and an uncompressed subannular anchor, according to the invention.

FIG. 71 is an illustration of a side perspective view of an orthogonally deliverable transcatheter heart valve with channel-less pre-determined anchor pathway(s) extending from cuff/collar through the valve body to the subannular/lower side, and distal right ventricular outflow tract (RVOT) tab, a proximal tab according to the invention according to the invention, with anchor delivery catheter, having sub-annular anchor connected by flexible tether to expandable tether lock, prior to cinching the lock down onto the collar surface and the tab against a lower portion of the valve body.

FIG. 72 is an illustration of a side perspective view of an orthogonally deliverable transcatheter heart valve with channel-less pre-determined anchor pathway(s) extending from cuff/collar through the valve body to the subannular/lower side, and distal right ventricular outflow tract (RVOT) tab, a proximal tab according to the invention according to the invention, with anchor delivery catheter, having sub-annular anchor connected by flexible tether to expandable tether lock, after cinching the lock down onto the collar surface and the tab against a lower portion of the valve body.

FIG. 73 is an illustration of a side view of a tether lock that is compressed within a delivery sheath (catheter), with the tether lock having a central aperture, with the braided tether threaded thru the central aperture of the tether lock so that the tether lock can slide along the tether.

FIG. 74 is an illustration of a side view of a tether lock that is released by withdrawal of the sheath into an expanded, uncompressed configuration, with the expanded tether lock having a central aperture, and with the braided tether threaded thru the central aperture of the tether lock, and the central aperture having one or more locking teeth to tighten onto the tether and prevent further sliding along the tether. The locking teeth can be a curved, one-way type of pawl or tooth, or they may be piercing teeth that engage the tether when the tether lock is expanded.

FIG. 75 is an illustration of a cross-sectional side view of a tether lock that is released by withdrawal of the sheath into an expanded, uncompressed configuration, with the expanded tether lock having a central aperture, and the central aperture having one or more locking teeth to tighten onto the tether and prevent further sliding along the tether. The locking teeth can be a curved, one-way type of pawl or tooth, or they may be piercing teeth that engage the tether when the tether lock is expanded.

FIG. 76 is an illustration of another preferred embodiment of the invention and shows a compressible valve having a P anchor (near posterior leaflet) and an A anchor (near anterior leaflet), with the tissue anchor loops attached to flexible elongated straps or rods similar to a cable tie, the flexible elongated strap having an attached anchor lock that engages with a section of the strap having teeth, the anchor lock forming an encircling head with a pawl in the head that engages the teeth of the strap, such that when the anchor lock/head slides down the flexible elongated strap, the anchor lock/head is locked into place when the pawl prevents the anchor lock from sliding up the strap.

FIG. 77 is an illustration of a side view of a tissue anchor loop attached to a strap with anchor lock slid into a lower, locked position.

FIG. 78 is an illustration of a side perspective view towards a septal leaflet side of a tricuspid valve replacement with an upper collar attached to a body portion, with an RVOT tab, a proximal tab, a P anchor and an A anchor, with anchor locks slid down into a lower locking position.

FIG. 79 is an illustration of a side perspective view from a septal leaflet side of a tricuspid valve replacement with an upper collar attached to a body portion, with an RVOT tab, a proximal tab, a P anchor and an A anchor, with anchor locks slid down into a lower locking position.

FIG. 80 is an illustration of a top view of a tricuspid valve replacement with an upper collar attached to a body portion, with an RVOT tab, a proximal tab, a P anchor and an A anchor, with anchor locks slid down into a lower locking position.

FIG. 81 is an illustration of a side perspective view from above a septal leaflet side of a tricuspid valve replacement with an upper collar attached to a body portion, with an RVOT tab, a proximal tab, central flow control element (tri-leaflet valve), an S anchor (anchor near septal leaflet) and a P-S anchor (anchor near postero-septal leaflet commissure), with anchor locks slid down into a lower locking position.

FIG. 82 is an illustration of a top view of a three-anchor tricuspid valve replacement with an upper collar attached to a body portion, with an RVOT tab, a proximal tab, central flow control element (tri-leaflet valve), an S anchor (anchor near septal leaflet), a P anchor (anchor near posterior leaflet), and an A anchor (near anterior leaflet), with anchor locks slid down into a lower locking position.

FIG. 83 is an illustration of a top view of a four-anchor tricuspid valve replacement with an upper collar attached to a body portion, with an RVOT tab, a proximal tab, central flow control element (tri-leaflet valve), an S anchor, a P anchor, a P-S anchor, and an A anchor, with anchor locks slid down into a lower locking position.

FIG. 84 is an illustration of a side view of a series showing how an anchor can progress from (i) a stowed position within a delivery catheter sheath, (ii) an extended anchor position, (iii) an extended open position, (iv) a retracted position, and (v) a locked position, with an indicator of relative depths A-B-C referring to (A) above the annulus, (B) at or near the annular plane, and (C) below the annulus, according to the invention.

FIGS. 85A-85F is an illustration of side perspective view of the relation of the anchor(s) to native tissue during the anchoring-tissue capture process, with an indicator of relative depths A-B-C referring to (A) above the annulus, (B) at or near the annular plane, and (C) below the annulus, with anchor configurations including (a) compressed shown in FIG. 85A, (b) extended shown in FIG. 85B, (c) opened shown in FIG. 85C, (d) retracted shown in FIG. 85D, (e) locked shown in FIG. 85E, and (f) trimmed shown in FIG. 85F, according to the invention.

FIG. 86 is an illustration of a side view of an orthogonally compressed valve within a valve delivery catheter, with pre-attached folded over elongated straps and anchor locks and with advancement (towing) wire attached to the RVOT tab to pull the valve out of the sheath by applying force to the distal end, according to the invention.

FIG. 87 is an illustration of a side view of a valve partially expelled from the delivery catheter by the advancement (towing) wire, with pre-attached folded over elongated straps and anchor locks, according to the invention.

FIG. 88 is an illustration of a side view of a fully released valve, with pre-attached elongated straps and anchor locks released from the catheter sheath and in ready position for anchor deployment and tissue capture, according to the invention.

FIG. 89 is an illustration of a side perspective view of an anchor delivery catheter delivering an anchor to a radiomarker spot on the valve collar, according to the invention.

FIG. 90 is an illustration of a side perspective view of an anchor deployed to a sub-annular position, and a tether lock mounted on the tether above the valve collar ready for the tissue cinching step, according to the invention.

FIG. 91 is an illustration of a side perspective view of an anchor deployed to a sub-annular position, retracted to capture the chordae and part of the anterior leaflet, with the tether lock slid down the tether to the valve collar to lock the anchor and cinch the native tissue, according to the invention.

FIGS. 92A-92G are illustrations of a variety of anchor loops and tissue capture structures, according to the present invention.

### DETAILED DESCRIPTION OF THE INVENTION

The invention is directed to anchor channels and subannular anchors for a dual-tab transcatheter heart valve replacement that is a low profile, orthogonally delivered (side-delivered) implantable prosthetic heart valve having an ring-shaped or annular support frame, an inner 2-or 3-panel sleeve, an elongated sub-annular distal anchoring tab extending into the right ventricular outflow tract, an elongated sub-annular proximal anchoring tab extending into the proximal sub-annular space, preferably between the anterior and the posterior leaflets.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Rather, these embodiments are provided so that this disclosure will be thorough and complete, and will fully convey the scope of the invention to those skilled in the art. Like numbers refer to like elements throughout. As used herein the term “and/or” includes any and all combinations of one or more of the associated listed items.

The terminology used herein is for the purpose of describing particular embodiments only and is not intended to limit the full scope of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal subparts. As will be understood by one skilled in the art, a range includes each individual member.

### DEFINITIONS

### Integrated Anchor Channel

In the description and claims herein, the term “integrated anchor channel”, “channel”, “anchor delivery channel”, or “anchor delivery conduit” is used to describe a cylindrical tube or similar tubular structure that is from 5-35 mm in length having a diameter of 2-10 mm that is attached to (exterior) or extends through (interior) the body and/or collar portion of the prosthetic valve. The channel has, in some embodiments, at least one radio-opaque marker or radio-opaque material or structure so that the location of the channel can be visualized with a suitable imaging modality (i.e. a modality under which the radio-opaque marker or material is easily differentiated from other components of the prosthetic valve and surrounding tissue) and an anchor delivery system catheter can be guided through the body of a patient to the site where the valve is mounted or to be mounted and specifically to the location of the channel. In one embodiment, delivery of an orthogonal valve is (length-wise delivery, height-and width-compression) through the femoral vein to the inferior vena cava (IVC) to the right atrium of the heart for anchoring the prosthetic tricuspid heart valve replacement, followed by IVC delivery of the anchoring system to install the subannular anchors.

### Orthogonal

In the description and claims herein, the term “orthogonal” is used to describe that the valves of the present invention are compressed and side-delivered at a roughly 90 degree angle compared to traditional iris-opening transcatheter heart valves. Traditional valves are iris-ing and have a central cylinder axis that is parallel to the length-wise axis of the delivery catheter and are deployed from the end of the delivery catheter in a manner akin to pushing a closed umbrella out of a sleeve. The valves of the present invention are compressed and delivered in a sideways manner. Traditional iris-ing valves can only be expanded as large as what the internal diameter of the delivery catheter will allow. Efforts to increase the expanded diameter of traditional valves have run into the problems of trying to compress too much material and structure into too little space.

Mathematically, the term orthogonal refers to an intersecting angle of 90 degrees between two lines or planes. As used, herein the term “substantially orthogonal” refers to an intersecting angle ranging from 75 to 105 degrees. The intersecting angle or orthogonal angle refers to both (i) the relationship between the lengthwise cylindrical axis of the delivery catheter and the long-axis of the compressed valve of the invention, where the long-axis is perpendicular to the central cylinder axis of traditional valves, and (ii) the relationship between the long-axis of the compressed or expanded valve of the invention and the axis defined by the blood flow through the prosthetic heart valve where the blood is flowing, e.g. from one part of the body or chamber of the heart to another downstream part of the body or chamber of the heart, such as from an atrium to a ventricle through a native annulus.

### Transcatheter

In the description and claims herein, the term “transcatheter” is used to define the process of accessing, controlling, and delivering a medical device or instrument within the lumen of a catheter that is deployed into a heart chamber, as well as an item that has been delivered or controlled by such as process. Transcatheter access is known to include via femoral artery and femoral vein, via brachial artery and vein, via carotid and jugular, via intercostal (rib) space, and via sub-xyphoid. Transcatheter can be synonymous with transluminal and is functionally related to the term “percutaneous” as it relates to delivery of heart valves.

In some embodiments of the invention, the transcatheter approach includes (i) advancing to the tricuspid valve or pulmonary artery of the heart through the inferior vena cava via the femoral vein, (ii) advancing to the tricuspid valve or pulmonary artery of the heart through the superior vena cava via the jugular vein, (iii) advancing to the mitral valve of the heart through a trans-septal approach, e.g. fossa ovalis or lower, via the IVC-femoral or the SVC-jugular approach.

### Annular Support Frame

In the description and claims herein, the term “annular support frame”, and also “wire frame” or “flange or “collar” refers to a three-dimensional structural component that is seated within a native valve annulus and is used as a mounting element for a leaflet structure, a flow control component, or a flexible reciprocating sleeve or sleeve-valve.

In some embodiments, the annular support frame is a self-expanding annular support frame, having a central channel and an outer perimeter wall circumscribing a central vertical axis in an expanded configuration. The perimeter wall encompasses both the collar and the lower body portions.

The perimeter wall can be further defined as having a front wall portion and a back wall portion, which are connected along a near side (to the IVC) or proximal side to a proximal fold area, and connected along a far or distal side to a distal fold area.

This front wall portion can be further defined as having a front upper collar portion and a front lower body portion, and the back wall portion can be further defined as having a back upper collar portion and a back lower body portion.

The annular support frame has a flow control component mounted within the annular support frame and configured to permit blood flow in a first direction through an inflow end of the valve and block blood flow in a second direction, opposite the first direction, through an outflow end of the valve.

Since the frame is preferably made of superelastic metal or alloy such as Nitinol, the frame is compressible. Preferably, the frame is constructed of a plurality of compressible wire cells having an orientation and cell geometry substantially orthogonal to the central vertical axis to minimize wire cell strain when the annular support frame when configured in a vertical compressed configuration, a rolled compressed configuration, or a folded compressed configuration.

### Annular Support Frame Structure

The annular support frame can be a ring, or cylindrical or conical tube, made from a durable, biocompatible structural material such as Nitinol or similar alloy, wherein the annular support frame is formed by manufacturing the structural material as a braided wire frame, a laser-cut wire frame, or a wire loop. The annular support frame may be about 5-60 mm in height, may have an outer diameter dimension, R, of 30-80 mm, and an inner diameter dimension of 31-79 mm, accounting for the thickness of the wire material itself. As stated, the annular support frame can have a side-profile of a ring shape, cylinder shape, conical tube shape, but may also have a side profile of a flat-cone shape, an inverted flat-cone shape (narrower at top, wider at bottom), a concave cylinder (walls bent in), a convex cylinder (walls bulging out), an angular hourglass, a curved, graduated hourglass, a ring or cylinder having a flared top, flared bottom, or both. In some embodiments, the annular support frame used in the prosthetic heart valve deployed in the tricuspid annulus may have a complex shape determined by the anatomical structures where the valve is being mounted. For example, in the tricuspid annulus, the circumference of the tricuspid valve may be a rounded ellipse, the septal wall is known to be substantially vertical, and the tricuspid is known to enlarge in disease states along the anterior-posterior line. Accordingly, a prosthetic heart valve may start in a roughly tubular configuration, and be heat-shaped to provide an upper atrial cuff or flange for atrial sealing and a lower trans-annular tubular or cylindrical section having an hourglass cross-section for about 60-80% of the circumference to conform to the native annulus along the posterior and anterior annular segments while remaining substantially vertically flat along 20-40% of the annular circumference to conform to the septal annular segment.

### Annular Support Frame Covering

The annular support frame is optionally internally or externally covered, partially or completely, with a biocompatible material such as pericardium. The annular support frame may also be optionally externally covered, partially or completely, with a second biocompatible material such as polyester or Dacron®.

### Annular Support Frame Purpose

The annular support frame has a central axial lumen where a prosthetic heart valve or flow-control structure, such as a reciprocating compressible sleeve, is mounted across the diameter of the lumen. The annular support frame is also tensioned against the inner aspect of the native annulus and provides structural patency to a weakened annular ring.

### Annular Support Frame Optional Collars

The annular support frame may optionally have a separate atrial collar attached to the upper (atrial) edge of the frame, for deploying on the atrial floor, that is used to direct blood from the atrium into the sleeve and to seal against blood leakage around the annular support frame. The annular support frame may also optionally have a separate ventricular collar attached to the lower (ventricular) edge of the frame, for deploying in the ventricle immediately below the native annulus that is used to prevent regurgitant leakage during systole, to prevent dislodging of the device during systole, to sandwich or compress the native annulus or adjacent tissue against the atrial collar, and optionally to attach to and support the sleeve/conduit.

### Annular Support Frame Delivery

The annular support frame may be compressed for transcatheter delivery and may be expandable as a self-expandable shape-memory element or using a transcatheter expansion balloon. Some embodiments may have both an atrial collar and a ventricular collar, whereas other embodiments within the scope of the invention include prosthetic heart valves having either a single atrial collar, a single ventricular collar, or having no additional collar structure.

### Frame Material

Preferably, the frame is made from a superelastic metal component, such as laser-cut Nitinol tube, or flat sheet or other similarly functioning material such as braided wire. The material may be used for the frame/stent, for the collar, and/or for anchors. It is contemplated as within the scope of the invention to use other shape memory alloys, as well as polymer composites including composites containing carbon nanotubes, carbon fibers, metal fibers, glass fibers, and polymer fibers. It is contemplated that the frame may be constructed as a braid, wire, or laser cut frame. Laser cut frames are preferably made from Nitinol, but also without limitation made from stainless steel, cobalt chromium, titanium, and other functionally equivalent metals and alloys.

One key aspect of the frame design is that it be compressible and when released have the stated property that it returns to its original (uncompressed) shape. This requirement limits the potential material selections to metals and plastics that have shape memory properties. With regards to metals, Nitinol has been found to be especially useful since it can be processed to be austenitic, martensitic or super elastic. Martensitic and super elastic alloys can be processed to demonstrate the required mechanical behavior.

### Laser Cut

One possible construction of the wire frame envisions the laser cutting of a thin, isodiametric Nitinol tube. The laser cuts form regular cutouts in the thin Nitinol tube. In one embodiment, the Nitinol tube expands to form a three-dimensional structure formed from diamond-shaped cells. The structure may also have additional functional elements, e.g. loops, anchors, etc. for attaching accessory components such as biocompatible covers, tissue anchors, releasable deployment and retrieval control guides, knobs, attachments, rigging, and so forth.

Secondarily the tube is thermo-mechanically processed using industry standard Nitinol shape forming methods. The treatment of the wire frame in this manner will form a device that has shape memory properties and will readily revert to the memory shape once deployed.

### Braided Wire

Another possible construction of the wire frame envisions utilizing simple braiding techniques using a Nitinol wire and a simple braiding fixture. The wire is wound on the braiding fixture in a pattern until an isodiametric tube is formed. Secondarily, the braided wire frame is placed on a shaping fixture and processed using industry standard Nitinol shape forming methods.

### Flow Control Component

In the description and claims herein, the term “flow control component” refers in a non-limiting sense to a leaflet structure having 2-, 3-, 4-leaflets of flexible biocompatible material such a treated or untreated pericardium that is sewn or joined to a annular support frame, to function as a prosthetic heart valve. Such a valve can be a heart valve, such as a tricuspid, mitral, aortic, or pulmonary, that is open to blood flowing during diastole from atrium to ventricle, and that closes from systolic ventricular pressure applied to the outer surface. Repeated opening and closing in sequence can be described as “reciprocating.”

### Tissue Anchor

In the description and claims herein, the term “tissue anchor” or “plication tissue anchor” or “secondary tissue anchor”, or “dart” or “pin” refers to a fastening device that connects the upper atrial frame to the native annular tissue, usually at or near the periphery of the collar. The anchor may be positioned to avoid piercing tissue and just rely on the compressive force of the two plate-like collars on the captured tissue, or the anchor, itself or with an integrated securement wire, may pierce through native tissue to provide anchoring, or a combination of both. The anchor may have a specialized securement mechanism, such as a pointed tip with a groove and flanged shoulder that is inserted or popped into a mated aperture or an array of mated apertures that allow the anchor to attach, but prevent detachment when the aperture periphery locks into the groove near the flanged shoulder. The securement wire may be attached or anchored to the collar opposite the pin by any attachment or anchoring mechanisms, including a knot, a suture, a wire crimp, a wire lock having a cam mechanism, or combinations.

As a point of further definition, the term “expandable” is used herein to refer to a component of the heart valve capable of expanding from a first, delivery diameter to a second, implantation diameter. An expandable structure, therefore, does not mean one that might undergo slight expansion from a rise in temperature, or other such incidental cause. Conversely, “non-expandable” should not be interpreted to mean completely rigid or a dimensionally stable, as some slight expansion of conventional “non-expandable” heart valves, for example, may be observed.

### Prosthetic Heart Valve

The term prosthesis or prosthetic encompasses both complete replacement of an anatomical part, e.g. a new mechanical valve replaces a native valve, as well as medical devices that take the place of and/or assist, repair, or improve existing anatomical parts, e.g. native valve is left in place. For mounting within a passive assist cage, the invention contemplates a wide variety of (bio) prosthetic artificial heart valves. Contemplated as within the scope of the invention are ball valves (e.g. Starr-Edwards), bileaflet valves (St. Jude), tilting disc valves (e.g. Bjork-Shiley), stented pericardium heart-valve prosthesis' (bovine, porcine, ovine) (Edwards line of bioprostheses, St. Jude prosthetic heart valves), as well as homograft and autograft valves. For bioprosthetic pericardial valves, it is contemplated to use bioprosthetic aortic valves, bioprosthetic mitral valves, bioprosthetic tricuspid valves, and bioprosthetic pulmonary valves.

### Tethers

The tethers are made from surgical-grade materials such as biocompatible polymer suture material. Non-limiting examples of such material include ultra-high-molecular weight polyethylene (UHMWPE), 2-0 exPFTE (polytetrafluoroethylene) or 2-0 polypropylene. In one embodiment the tethers are inelastic. It is also contemplated that one or more of the tethers may optionally be elastic to provide an even further degree of compliance of the valve during the cardiac cycle.

### Tines-Anchors-Tines/Barbs

The device can be seated within the valvular annulus through the use of tines or barbs. These may be used in conjunction with, or in place of one or more tethers. The tines or barbs are located to provide attachment to adjacent tissue. Tines are forced into the annular tissue by mechanical means such as using a balloon catheter. In one non-limiting embodiment, the tines may optionally be semi-circular hooks that upon expansion of the wire frame body, pierce, rotate into, and hold annular tissue securely. Anchors are deployed by over-wire delivery of an anchor or anchors through a delivery catheter. The catheter may have multiple axial lumens for delivery of a variety of anchoring tools, including anchor setting tools, force application tools, hooks, snaring tools, cutting tools, radio-frequency and radiological visualization tools and markers, and suture/thread manipulation tools. Once the anchor(s) are attached to the moderator band, tensioning tools may be used to adjust the length of tethers that connect to an implanted valve to adjust and secure the implant as necessary for proper functioning. It is also contemplated that anchors may be spring-loaded and may have tether-attachment or tether-capture mechanisms built into the tethering face of the anchor(s). Anchors may also have in-growth material, such as polyester fibers, to promote in-growth of the anchors into the myocardium.

In one embodiment, where a prosthetic heart valve may or may not include a ventricular collar, the anchor or dart is not attached to a lower ventricular collar, but is attached directly into annular tissue or other tissue useful for anchoring.

Tube and/or Cover Material-Biological Tissue

The tissue used herein is a biological tissue that is a chemically stabilized pericardial tissue of an animal, such as a cow (bovine pericardium) or sheep (ovine pericardium) or pig (porcine pericardium) or horse (equine pericardium). Preferably, the tissue is bovine pericardial tissue. Examples of suitable tissue include that used in the products Duraguard®, Peri-Guard®, and Vascu-Guard®, all products currently used in surgical procedures, and which are marketed as being harvested generally from cattle less than 30 months old. Other patents and publications disclose the surgical use of harvested, biocompatible animal thin tissues suitable herein as biocompatible “jackets” or sleeves for implantable stents, including for example, U.S. Pat. No. 5,554,185 to Block, U.S. Patent No. 7,108,717 to Design & Performance-Cyprus Limited disclosing a covered stent assembly, U.S. Pat. No. 6,440,164 to Scimed Life Systems, Inc. disclosing a bioprosthetic heart valve for implantation, and U.S. Pat. No. 5,336,616 to LifeCell Corporation discloses acellular collagen-based tissue matrix for transplantation.

### Polymers

In one embodiment, the conduit may optionally be made from a synthetic material such a polyurethane or polytetrafluoroethylene. Where a thin, durable synthetic material is contemplated, e.g. for a covering, synthetic polymer materials such expanded polytetrafluoroethylene or polyester may optionally be used. Other suitable materials may optionally include thermoplastic polycarbonate urethane, polyether urethane, segmented polyether urethane, silicone polyether urethane, silicone-polycarbonate urethane, and ultra-high molecular weight polyethylene. Additional biocompatible polymers may optionally include polyolefins, elastomers, polyethylene-glycols, polyethersulphones, polysulphones, polyvinylpyrrolidones, polyvinylchlorides, other fluoropolymers, silicone polyesters, siloxane polymers and/or oligomers, and/or polylactones, and block co-polymers using the same.

### Polyamides (PA)

PA is an early engineering thermoplastic invented that consists of a “super polyester” fiber with molecular weight greater than 10,000. It is commonly called Nylon. Application of polyamides includes transparent tubing's for cardiovascular applications, hemodialysis membranes, and also production of percutaneous transluminal coronary angioplasty (PTCA) catheters.

### Polyolefin

Polyolefins include polyethylene and polypropylene are the two important polymers of polyolefins and have better biocompatibility and chemical resistance. In cardiovascular uses, both low-density polyethylene and high-density polyethylene are utilized in making tubing and housings. Polypropylene is used for making heart valve structures.

### Polyesters

Polyesters includes polyethylene-terephthalate (PET), using the name Dacron. It is typically used as knitted or woven fabric for vascular grafts. Woven PET has smaller pores which reduces blood leakage and better efficiency as vascular grafts compared with the knitted one. PET grafts are also available with a protein coating (collagen or albumin) for reducing blood loss and

better biocompatibility [39]. PET vascular grafts with endothelial cells have been searched as a means for improving patency rates. Moreover, polyesters are widely preferred material for the manufacturing of bioabsorbable stents. Poly-L-lactic acids (PLLA), polyglycolic acid (PGA), and poly(D, L-lactide/glycolide) copolymer (PDLA) are some of the commonly used bioabsorbable polymers.

### Polytetrafluoroethylene

Polytetrafluoroethylene (PTFE) is synthetic fluorocarbon polymer with the common commercial name of Teflon by Dupont Co. Common applications of PTFE in cardiovascular engineering include vascular grafts and heart valves. PTFE sutures are used in the repair of mitral valve for myxomatous disease and also in surgery for prolapse of the anterior or posterior leaflets of mitral valves. PTFE is particularly used in implantable prosthetic heart valve rings. It has been successfully used as vascular grafts when the devices are implanted in high-flow, large-diameter arteries such as the aorta. Problem occurs when it is implanted below aortic bifurcations and another form of PTFE called elongated-PTFE (e-PTFE) was explored. Expanded PTFE is formed by compression of PTFE in the presence of career medium and finally extruding the mixture. Extrudate formed by this process is then heated to near its glass transition temperature and stretched to obtain microscopically porous PTFE known as e-PTFE. This form of PTFE was indicated for use in smaller arteries with lower flow rates promoting low thrombogenicity, lower rates of restenosis and hemostasis, less calcification, and biochemically inert properties.

### Polyurethanes

Polyurethane has good physiochemical and mechanical properties and is highly biocompatible which allows unrestricted usage in blood contacting devices. It has high shear strength, elasticity, and transparency. Moreover, the surface of polyurethane has good resistance for microbes and the thrombosis formation by PU is almost similar to the versatile cardiovascular biomaterial like PTFE. Conventionally, segmented polyurethanes (SPUs) have been used for various cardiovascular applications such as valve structures, pacemaker leads and ventricular assisting device.

Covered Wire frame Materials

Drug-eluting wire frames are contemplated for use herein. DES basically consist of three parts: wire frame platform, coating, and drug. Some of the examples for polymer free DES are Amazon Pax (MINVASYS) using Amazonia CroCo (L605) cobalt chromium (Co-Cr) wire frame with Paclitaxel as an antiproliferative agent and abluminal coating have been utilized as the carrier of the drug. BioFreedom (Biosensors Inc.) using stainless steel as base with modified abluminal coating as carrier surface for the antiproliferative drug Biolimus A9. Optima (CID S.r.I.) using 316 L stainless steel wire frame as base for the drug Tacrolimus and utilizing integrated turbostratic carbofilm as the drug carrier. VESTA sync (MIV Therapeutics) using GenX stainless steel (316 L) as base utilizing microporous hydroxyapatite coating as carrier for the drug Sirolimus. YUKON choice (Translumina) used 316 L stainless steel as base for the drugs Sirolimus in combination with Probucol.

Biosorbable polymers may also be used herein as a carrier matrix for drugs. Cypher, Taxus, and Endeavour are the three basic type of bioabsorbable DES. Cypher (J&J, Cordis) uses a 316 L stainless steel coated with polyethylene vinyl acetate (PEVA) and poly-butyl methacrylate (PBMA) for carrying the drug Sirolimus. Taxus (Boston Scientific) utilizes 316 L stainless steel wire frames coated with translute Styrene Isoprene Butadiene (SIBS) copolymer for carrying Paclitaxel which elutes over a period of about 90 days. Endeavour (Medtronic) uses a cobalt chrome driver wire frame for carrying Zotarolimus with phosphorylcholine as drug carrier. BioMatrix employing S-Wire frame (316 L) stainless steel as base with polylactic acid surface for carrying the antiproliferative drug Biolimus. ELIXIR-DES program (Elixir Medical Corp) consisting both polyester and polylactide coated wire frames for carrying the drug Novolimus with cobalt-chromium (Co-Cr) as base. JACTAX (Boston Scientific Corp.) utilized D-lactic polylactic acid (DLPLA) coated (316 L) stainless steel wire frames for carrying Paclitaxel. NEVO (Cordis Corporation, Johnson & Johnson) used cobalt chromium (Co-Cr) wire frame coated with polylactic-co-glycolic acid (PLGA) for carrying the drug Sirolimus.

Examples of embodiments of the reciprocating pressure conduit valve include the following details and features.

### EXAMPLE

The transcatheter prosthetic heart valve may be percutaneously delivered using a transcatheter process via the femoral through the IVC, carotid, sub-xyphoid, intercostal access across the chest wall, and trans-septal to the mitral annulus through the fossa ovalis.

The device is delivered via catheter to the right or left atrium and is expanded from a compressed shape that fits with the internal diameter of the catheter lumen. The compressed valve is loaded external to the patient into the delivery catheter, and is then pushed out of the catheter when the capsule arrives to the atrium. The cardiac treatment technician visualizes this delivery using available imaging techniques such as fluoroscopy or ultrasound.

In some embodiments the valve self-expands upon release from the catheter since it is constructed in part from shape-memory material, such as Nitinol®, a nickel-titanium alloy, or a cobalt-chromium alloy, alloys used in biomedical implants.

In another embodiment, the valve may be constructed of materials that require balloon-expansion after the capsule has been ejected from the catheter into the atrium.

The atrial collar/frame and the flow control component are expanded to their functional diameter, as they are deployed into the native annulus, providing a radial tensioning force to secure the valve. Once the frame is deployed about the tricuspid annulus, fasteners secure the device about the native annulus. Additional fastening of the device to native structures may be performed, and the deployment is complete. Further adjustments using hemodynamic imaging techniques are contemplated as within the scope of the invention in order to ensure the device is secure, is located and oriented as planned, and is functioning as a substitute or successor to the native tricuspid valve.

### Example—Manufacturing Process

In some embodiments the invention includes a process for manufacturing an orthogonally delivered (side-delivered) transcatheter prosthetic heart valve frame, comprising:
(i) using additive or subtractive metal or metal-alloy manufacturing to produce a self-expanding annular support frame,

wherein the additive metal or metal-alloy manufacturing is 3D printing or direct metal laser sintering (powder melt), and

wherein the subtractive metal or metal-alloy manufacturing is photolithography, laser sintering/cutting, CNC machining, electrical discharge machining.

In other embodiments, there is provided a process for manufacturing an orthogonally delivered (side-delivered) transcatheter prosthetic heart valve frame, further comprising the steps of:
(ii) mounting a flow control component within the valve frame, said flow control component configured to permit blood flow along the central vertical axis through an inflow end of the flow control component and block blood flow through an outflow end of the valve, and
(iii) covering an outer surface of the valve frame with a pericardium material or similar biocompatible material.

### Example—Compression Methods

In other embodiments, there is provided a method of compressing, wherein the implantable prosthetic heart valve is rolled or folded into a compressed configuration using a step selected from the group consisting of:
(i) unilaterally rolling into a compressed configuration from one side of the annular support frame;
(ii) bilaterally rolling into a compressed configuration from two opposing sides of the annular support frame;
(iii) flattening the annular support frame into two parallel panels that are substantially parallel to the long-axis, and then rolling the flattened annular support frame into a compressed configuration; and
(iv) flattening the annular support frame along a vertical axis to reduce a vertical dimension of the valve from top to bottom.

### PARTS LIST

101 anchoring system
110 anchor hook
112 shaft portion
114 hook portion
116 cable mount
118 anchoring tabs or barbs
120 delivery catheter
122 anchor channel
124 elongated pusher cable
126 hook open configuration
128 hook closed configuration
130 radius of hook gap
132 barb open configuration
134 barb closed configuration
136 radius from shaft
138 inner diameter of channel
140 split shaft
142 second anchor hook
144 second anchor channel
146 side-delivered prosthetic transcatheter heart valve
148 lumen of anchor channel
150 tether mount on anchor hook portion
152 tether loop on shaft
154 tether
155 tether lock
156 valve body portion/outer perimeter wall
158 interior of valve body portion
160 exterior of valve body portion
162 polyester cylinder channel or polyester lining of channel
164 radio-opaque marker
166 distal tab
168 proximal tab
170 compressible wire cells of annular support frame
172 valve collar portion
174 biocompatible cover material
176 flow control component
178 leaflets
180 supports or ribs holding leaflets
182 upper anchoring tab
184 front wall
186 back wall
188 proximal fold area
190 distal fold area
192 delivery catheter for valve
194 guide wire
196 valve advancing tool/guide wire sheath
198 threading aperture on distal tab
199 piercing tool

### Drawings

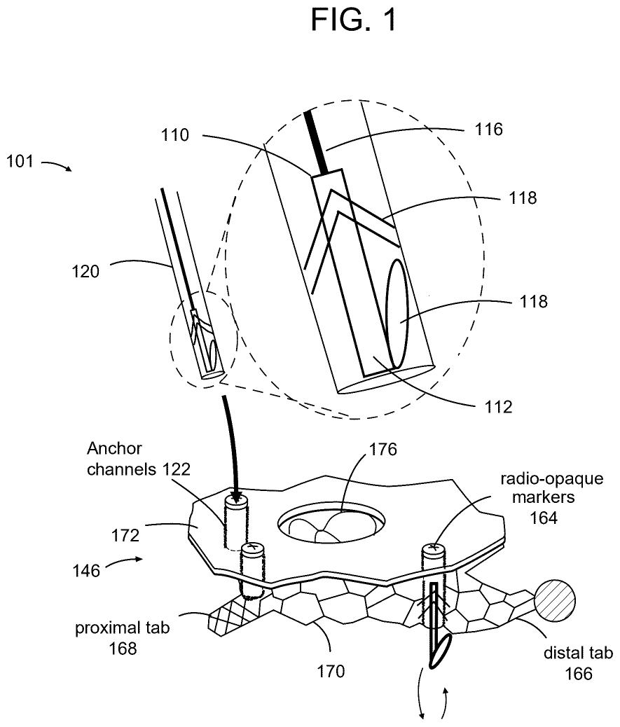

Referring now to the drawings, FIG. 1 is an illustration of an anchor hook 110 according to the invention. FIG. 1shows an anchor hook 110 disposed within the lumen of a delivery catheter 120 for delivery thru a selected location on the valve collar 172 for subannular actuation and deployment. FIG. 1 includes a detail illustration of the anchor hook 110 of FIG. 1and shows the collapsible/expandable anchor hook with downward hooking tabs or barbs 118 on the shaft portion 112 and shows the hook portion 114. In some embodiments, a tether 116 connects a tether mount on the hook portion to loop guide on the shaft portion for actuating the anchor hook from an open position to a closed position. FIG. 1 also shows an anchor hook 110 embodiment that is already mounted within an integral delivery conduit/channel 122 configured within the valve body 146. FIG. 1 also shows an orthogonally deliverable (side-deliverable) transcatheter heart valve 146 with integrated anchor conduit or channel(s) 122 extending from cuff/collar 172 through the valve body to the subannular/lower side, and distal right ventricular outflow tract (RVOT) tab 166, and a proximal tab 168, according to the invention.

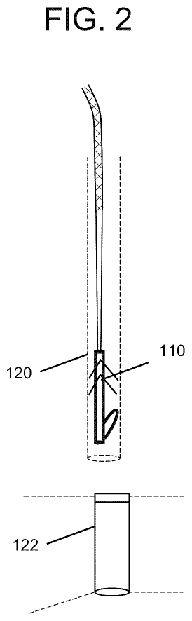

FIG. 2 is an illustration of a side view of an anchor delivery system according to the invention having a delivery catheter 120 sheathed over a compressed expandable anchor hook 110 attached to a pusher rod detachably connected to the anchor hook 110 for inserting into the channel 122

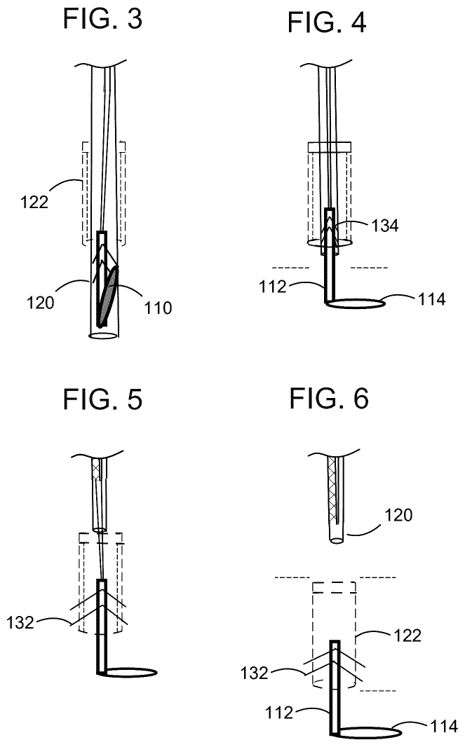

FIGS. 3-6 shows a process of (i) extending the anchor hook 110 to a subannular position, (ii) releasing the anchor hook 110 from a compressed configuration to an open, expanded configuration, and capturing subannular tissue such as chordae tendinea and/or leaflet and/or annular tissue, with the unfolded, opened anchor hook 114, optionally re-compressing or re-folding the anchor hook, with the tissue captured in the hook portion 114 and/or the hooking tabs, and/or optionally cinching the anchor hook and captured tissue upwards against the bottom subannular surface of the valve body, and (iii) releasing the barbs 132 extending from the shank by withdrawing the catheter 120, allowing the barbs 132 to lodge in or thru the cylindrical inner wall of the anchor channel 122.

FIG. 3 is an illustration of a step of a multi-part series showing a side view of a process of using an anchor hook 110 from a delivery catheter 120, with the anchor hook disposed within the delivery catheter, and the delivery catheter inserted thru the integrated anchor channel 122 in the valve, and with delivery catheter sheath at position 1.

FIG. 4 is an illustration of a step of a multi-part series showing a side view of a process of using an anchor hook from a delivery catheter, with the anchor hook partially disposed within the lumen of the delivery catheter, and partially expelled out of the lumen by partial removal of the delivery catheter sheath to position 2.

FIG. 5 is an illustration of a step of a multi-part series showing a side view of a process of using an anchor hook from a delivery catheter, with the bend portion 114 of the anchor hook expelled from the lumen by partial removal of the delivery catheter sheath to position 2, and with the anchor hook being adjusted/elongated to capture and secure native tissue or structures.

FIG. 6 is an illustration of a step of a multi-part series showing a side view of a process of using an anchor hook from a delivery catheter, with the anchor hook completely expelled from the lumen of the delivery catheter by withdrawal of the delivery catheter sheath to position 3, and compression and capture of the native tissue by adjusting or shortening the tether to close the anchor hook to a compressed configuration.

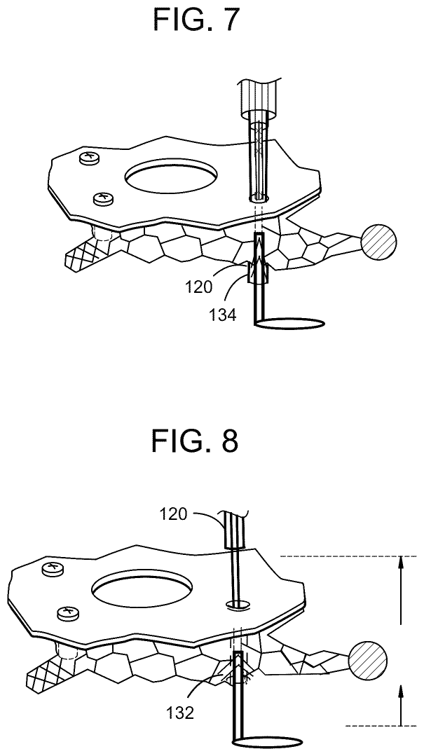

FIG. 7 is an illustration of a step of a series showing a side view of a process of expelling an anchor hook to a subannular position using a delivery catheter. Barbs 134 are in a compressed position.

FIG. 8 is an illustration of a step of a series showing a side view of a process of capturing native tissue by pulling the hook portion, and cinching subannular tissue/structures against the bottom edge of the valve body/channel, while unsheathing the previous compressed/flattened and now opened barbs 132 to lock the anchor hook in place by lodging into the mesh channel, with the anchor hook spanning the channel, and tightened to a tensioned configuration along the cinch axis.

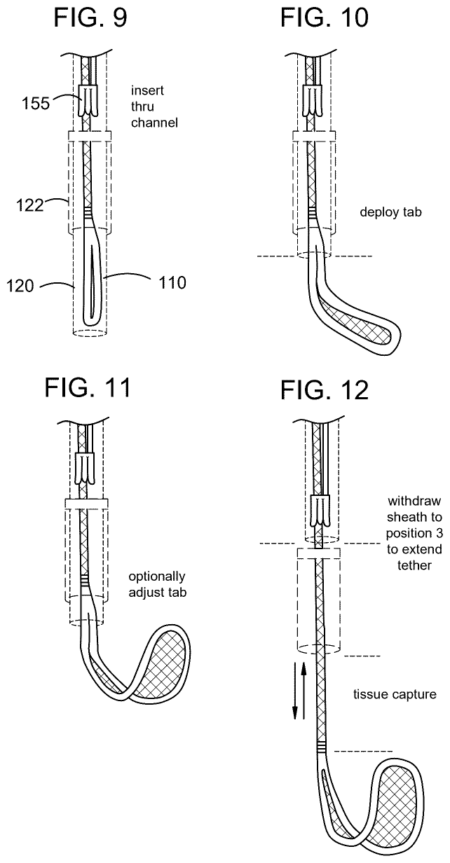

FIGS. 9-15 is a series of illustrations of another embodiment of the invention that uses a slidable lock 155 and tether instead of the expandable barbs to cinch and secure the anchor hook after the anchor hook has captured tissue.

FIG. 9 shows inserting the sheathed anchor hook 110 through the channel 122 to a subannular location.

FIG. 10 shows the catheter 120 partially withdrawn to expel the hook or bend portion to capture native tissue.

FIG. 11 show an optional step of adjusting or rotating the bend or hook portion.

FIG. 12 shows the sheath withdrawn to an atrial position while the hook portion is located in a subannular or ventricular position.

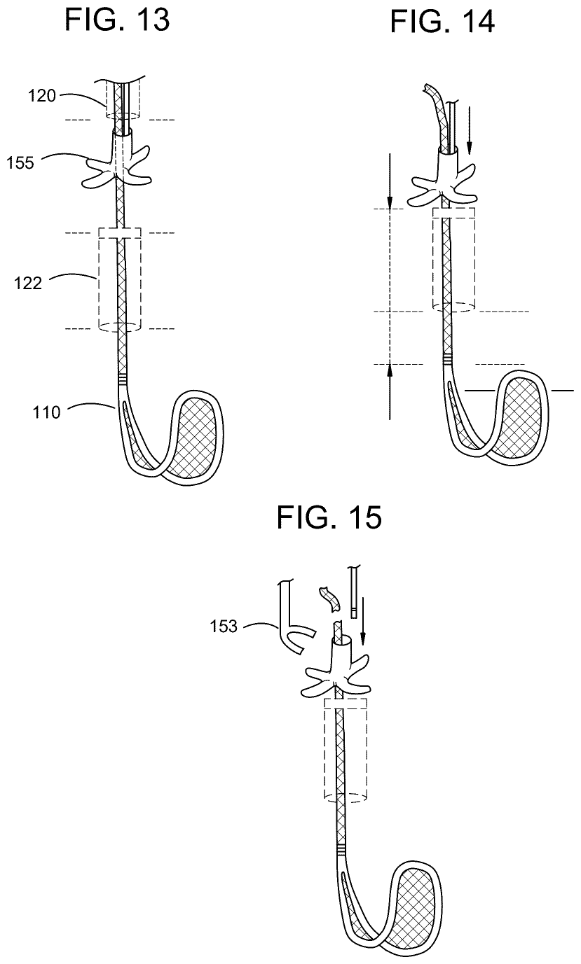

FIG. 13 shows the deployment of an expandable tether lock 155 that is expelled from the catheter 120 allowing it to expand to a size larger than the inner diameter of the channel 122.

FIG. 14 shows the tether pulling the hook portion and native tissue up into a capture position while pushing the tether lock down onto the top of the upper part of the channel.

FIG. 15 shows the tether being trimmed 153 to leave the cinched anchor hook in the prosthetic valve.

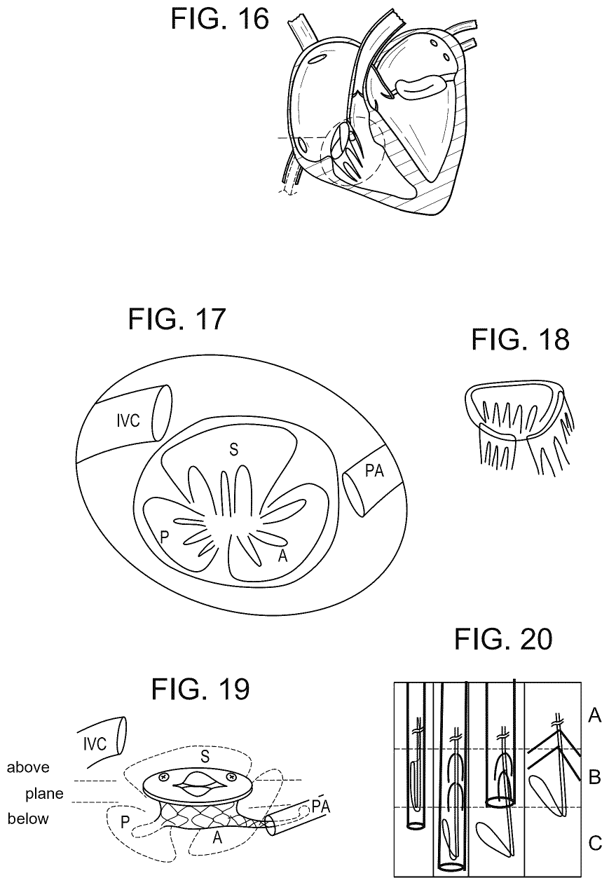

FIG. 16 is an illustration of a bisected view of a heart.

FIG. 17 is an illustration of a top view of a native tricuspid valve with location of inferior vena cava, location of pulmonary artery, and locations of the septal, posterior, and anterior leaflets.

FIG. 18 is an illustration of a side perspective view of a tricuspid leaflets in isolation.

FIG. 19 is an illustration of an orthogonal TVR valve 146 against illustrations of the location of native tissue, with IVC, PA, and the septal, posterior, and anterior leaflets, with an indicator of relative depths A-B-C referring to (A) above the annulus, (B) at or near the annular plane, and (C) below the annulus.

FIG. 20 is an illustration of a side view of a series showing how an anchor hook can progress from (i) a stowed position within a delivery catheter sheath/channel, (ii) to a subannular location while sheathed, (iii) to a subannular location with the anchor in an open position, and (iv) to a retracted position, according to the invention.

FIG. 21 is an illustration of a valve delivery process for an orthogonally delivered (side-delivered) valve, and shows a side view of a heart, with dashed circle showing a tricuspid region of interest.

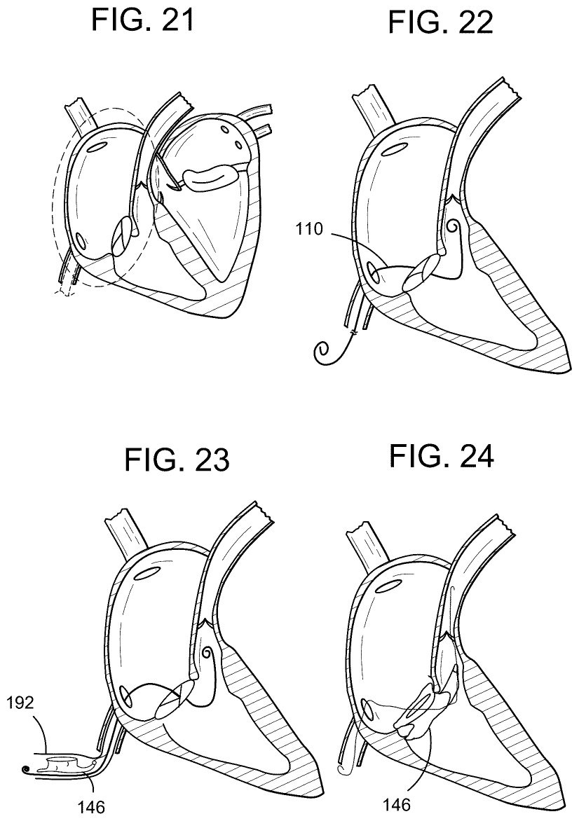

FIG. 22 is an illustration of a valve delivery process for an orthogonally delivered (side-delivered) valve, and shows a side view of a guide wire 194 step of a multi-step delivery process.

FIG. 23 is an illustration of a valve delivery process for an orthogonally delivered (side-delivered) valve, and shows a side view of a catheter delivery step of a multi-step delivery process, with a delivery catheter 192 containing an orthogonally compressed valve 146 delivered via the IVC.

FIG. 24 is an illustration of a valve delivery process for an orthogonally delivered (side-delivered) valve 146, and shows a side view of a valve deployment step of a multi-step delivery process, with a catheter expelling an expanded uncompressed valve partially into the tricuspid annulus, with RVOT tab placed in the pulmonary artery right ventricular outflow tract (RVOT) anchoring area.

FIG. 25 is an illustration of a valve delivery process for an orthogonally delivered (side-delivered) valve, and shows a side view of a catheter delivery step of a multi-step delivery process, with a TVR valve 146 fully seated within the tricuspid annulus, RVOT tab, and proximal tab positioned to secure the valve, and subannular anchor devices delivered and inserted into position on or in the valve.

FIG. 26 is an illustration of an anchor hook delivery process, and shows a side view TVR valve fully seated within the tricuspid annulus, RVOT tab, and proximal tab positioned to secure the valve, and subannular anchor devices 110 extended subannularly to capture native tissue, e.g. annular ring, chordae, and/or leaflet.

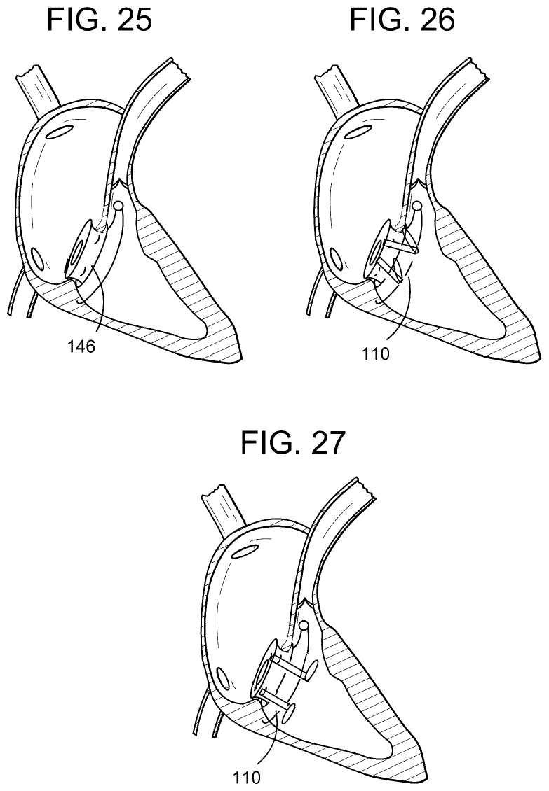

FIG. 27 is an illustration of an anchor hook delivery process, and shows a side view of a TVR valve fully seated within the tricuspid annulus, RVOT tab, and proximal tab positioned to secure the valve, and subannular anchor devices 110 retracted after capture of native tissue, e.g. annular ring, chordae, and/or leaflet.

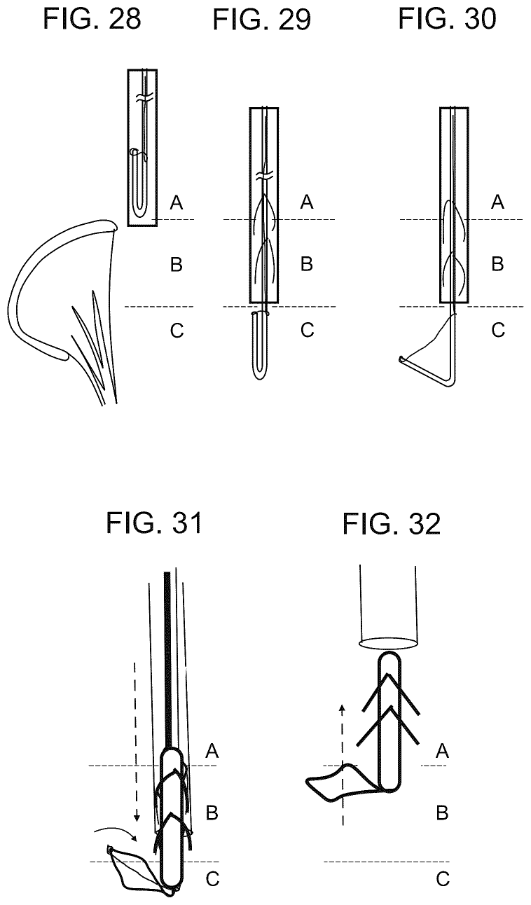

FIG. 28 is an illustration of side perspective view of a stowed or compressed anchor hook relative to native tissue during the anchoring-tissue capture process, with an indicator of relative depths A-B-C referring to (A) above the annulus.

FIG. 29 is an illustration of side perspective view of a partially expelled hook portion relative to native tissue during the anchoring-tissue capture process, with an indicator of relative depths A-B-C referring to (B) at or near the annular plane.

FIG. 30 is an illustration of side perspective view of an opened hook or bend portion of an anchor hook relative to native tissue during the anchoring-tissue capture process, with an indicator of relative depths A-B-C referring to (C) below the annulus.

FIG. 31 is an illustration of an anchor hook that has been adjusted from a compressed configuration, to an extended/opened configuration to capture native tissue.

FIG. 32 is an illustration of an anchor hook that has been adjusted from an open position to a retracted, and/or cinched configuration, with the sheath removed to allow the barbs to lodge into the channel wall.

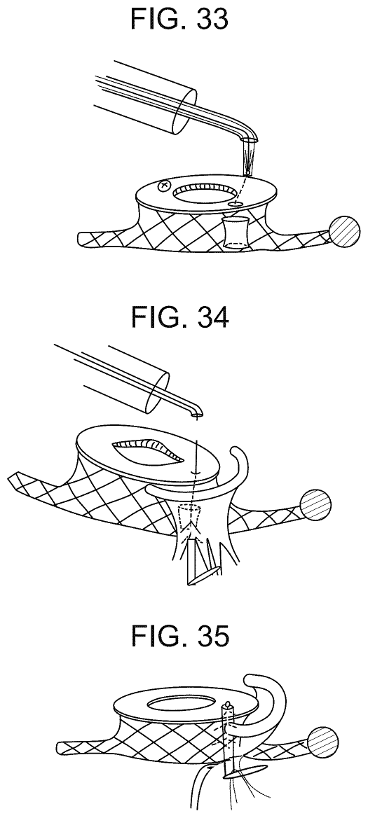

FIG. 33 is an illustration of a side perspective view of an anchor delivery catheter delivering an anchor hook to a radiomarker spot on the valve collar, according to the invention.

FIG. 34 is an illustration of a side perspective view of an anchor hook deployed to a sub-annular position, according to the invention.

FIG. 35 is an illustration of a side perspective view of an anchor hook that has been deployed to a sub-annular position, and then retracted to capture the chordae and part of the anterior leaflet, according to the invention.

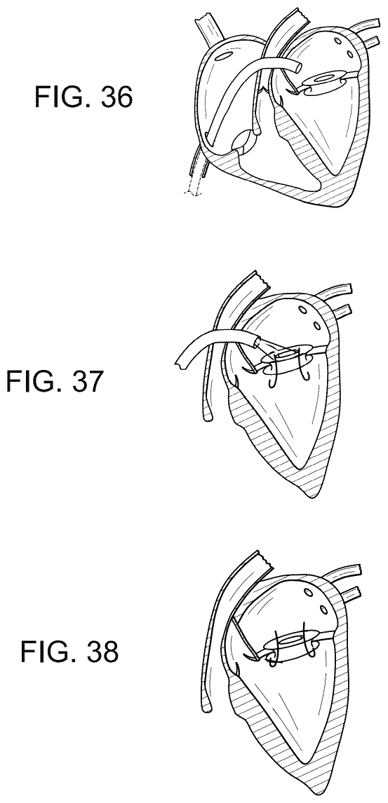

FIG. 36 is an illustration of a side perspective view a transcatheter delivery catheter that has access to the mitral valve from the IVC thru a trans-septal access to deliver an orthogonally deliverable (side-deliverable) mitral valve replacement prosthesis.

FIG. 37 is an illustration of a side perspective view a mitral valve embodiment having the anchor hooks deployed to an expanded position, according to the invention.

FIG. 38 is an illustration of a side perspective view a mitral valve embodiment having the anchor hooks compressed and/or cinched to capture native tissue, according to the invention.

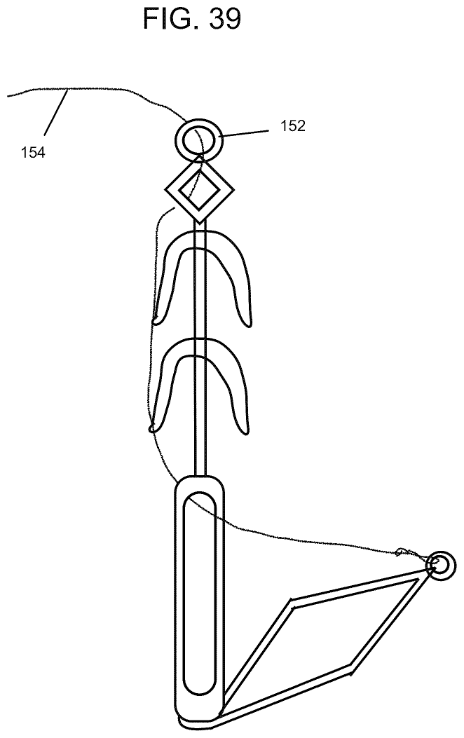

FIG. 39 is an illustration of one embodiment of an anchor hook according to the invention. FIG. 39 shows from the top, a proximal tether loop 152, a single diamond-shaped cell, a series of three anchoring tabs or barbs disposed along a central shaft portion, and a hook portion attached at a distal end of the central shaft portion, where the hook portion is shown in an extended configuration. The extend hook portion configuration allows the anchor hook to have a catheter sheath withdrawn, or have the hook portion expelled from a channel, and the shape memory aspect of the hook portion will curl as it is it expelled from the cylinder lumen. Following capture of the native tissue by the self-curling hook portion, the anchoring tabs or barbs can be exposed by withdrawing a sheath further or expelling the device further out of the channel. The anchoring tabs or barbs can be pushed into native tissue, pushed into an outer polyester covering, or both, to further secure the valve within the native annulus.

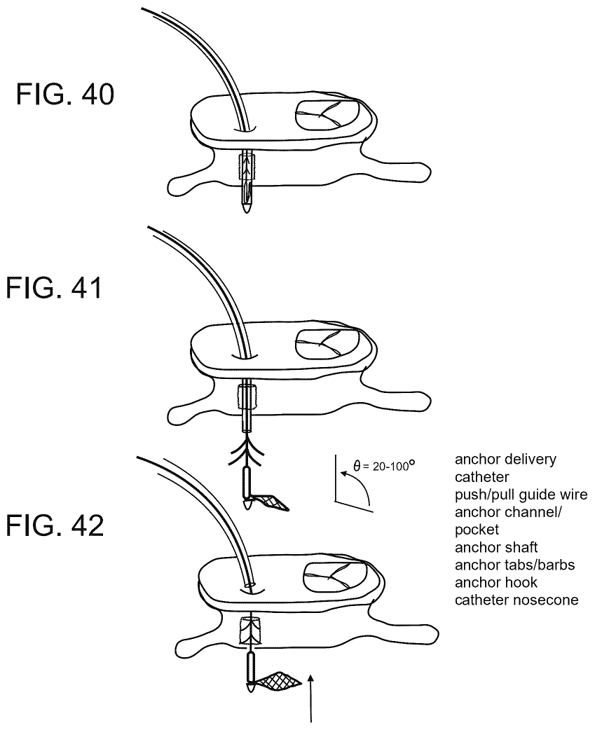

FIG. 40 is an illustration of one embodiment of an anchor hook disposed within a delivery catheter that extends through an anchor channel in the valve body to a subannular tissue-capture position, according to the invention. The anchoring tabs or barbs and the hook portion are initially compressed against the shaft portion when within the delivery catheter. When the delivery catheter is advanced thru an anchor channel to a subannular position, an elongated pusher cable that is attached to a proximal end of the shaft portion unsheathes the anchor hook and expands the anchoring tabs or barbs and the hook portion to a heat-set shape-memory configuration.

FIG. 41 is an illustration of one embodiment of an anchor hook released from the delivery catheter allowing the shape-memory material to expand with anchoring tabs or barbs extending laterally, and hook portion extending away from the shaft portion, according to the invention. With the hook portion in a position to capture a leaflet and/or chordae tendinea, the pusher cable is then pulled in a proximal direction so that the hook portion pulls the captured tissue against the bottom of the valve body or against the annular ring tissue.

FIG. 42 is an illustration of one embodiment of an anchor hook partially pulled back into the anchor channel with anchoring tabs or barbs compressed into the lumen of the anchor channel, and lower shaft portion and hook portion remaining outside of the anchor channel for capture and securement/anchoring of native tissue to the valve body, according to the invention.

Pulling the pusher cable also draws the tabs/barbs into the anchor channel, where the channel is narrower than the extended radius of the tabs/barbs. This causes the tabs/barbs to partially fold toward the shaft portion, and create a tensioning force with each tab/barb pressing against the interior surface of the channel. Where the channel is a mesh or braid, the tabs/barbs are forced into the channel wall. The direction of the barb also inhibits distal movement of the anchor shaft, i.e. inhibits movement of the anchor away from the valve body.

### Retrievability

The retrievability of the anchor hook originates from the ability of the delivery catheter to slide over the shaft portion while the tabs/barbs are engaging the channel wall, and to flatten the tabs/barbs back towards or against the shaft portion, thus disengaging the tabs/barbs from the channel interior wall. Once the tabs/barbs are disengaged, the delivery catheter is advanced distally to a subannular/ventricular position to release the hook portion from the captured native tissue. Once the hook portion is freed from the captured tissue, a tether that is attached to the anchor hook is pulled and folds the hook portion against the shaft portion. Once the hook portion is folded against the shaft portion, the entire anchor hook is then pulled into the delivery catheter and sheathed by the delivery catheter for withdrawal and/or redeployment of the anchor.

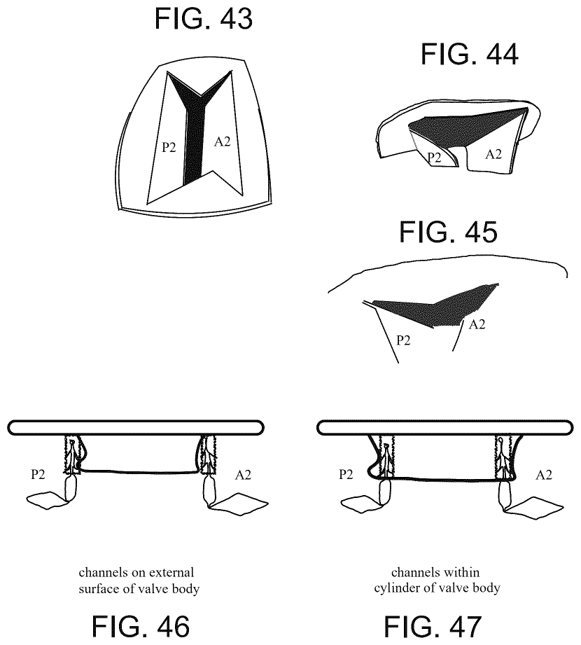

FIG. 43 is an illustration of a native mitral valve from a top perspective view above the annular plane from a trans-septal access side view, and shows the A2 leaflet and P2 leaflet in a partially open position.

FIG. 44 is an illustration of a native mitral valve from a bottom perspective view below the annular plane from a P2 side view, and shows the A2 leaflet and P2 leaflet in a partially open position.

FIG. 45 is an illustration of a native mitral valve from a bottom perspective view below the annular plane from an A2 side view, and shows the A2 leaflet and P2 leaflet in a partially open position.

FIG. 46 is an illustration of a prosthetic transcatheter heart valve from a plan or side view, and shows a pair of anchor hooks, each disposed within an anchor channel on the exterior surface of the valve body. A first anchor hook in an exterior channel is shown with hook portion extended to capture a P2 mitral leaflet, and a second anchor hook in an exterior channel is shown with hook portion extended to capture a A2 mitral leaflet.

The anchoring tabs or barbs and the hook portion are initially compressed against the shaft portion when within the delivery catheter. When the delivery catheter is advanced thru an anchor channel to a subannular position, where an elongated pusher cable that is attached to a proximal end of the shaft portion unsheathes the anchor hook and expands the anchoring tabs or barbs and the hook portion to a heat-set shape-memory configuration. With the hook portion in a position to capture an A2 or P2 leaflet and/or chordae tendinea, the pusher cable is then pulled in a proximal direction so that the hook portion pulls the captured tissue against the bottom of the valve body or against the annular ring tissue.

Pulling the pusher cable also draws the tabs/barbs into the anchor channel, where the channel is narrower than the extended radius of the tabs/barbs. This causes the tabs/barbs to partially fold toward the shaft portion, and create a tensioning force with each tab/barb pressing against the interior surface of the channel. Where the channel is a mesh or braid, the tabs/barbs are forced into the channel wall.

The retrievability of the anchor hook originates from the ability of the delivery catheter to slide over the shaft portion while the tabs/barbs are engaging the channel wall, and to flatten the tabs/barbs back towards or against the shaft portion, thus disengaging the tabs/barbs from the channel interior wall. Once the tabs/barbs are disengaged, the delivery catheter is advanced distally to a subannular/ventricular position to release the hook portion from the captured native tissue. Once the hook portion is freed from the captured tissue, a tether that is attached to the anchor hook is pulled and folds the hook portion against the shaft portion. Once the hook portion is folded against the shaft portion, the entire anchor hook is then pulled into the delivery catheter and sheathed by the delivery catheter for withdrawal and/or redeployment of the anchor.

FIG. 47 is an illustration of a prosthetic transcatheter heart valve from a plan or side view, and shows a pair of anchor hooks, each disposed within an anchor channel within the cylinder of the valve body. A first anchor hook is shown with hook portion extended to capture a P2 mitral leaflet, and a second anchor hook is shown with hook portion extended to capture a A2 mitral leaflet.

FIG. 48 is an illustration of an anchor in a stowed or compressed delivery position.

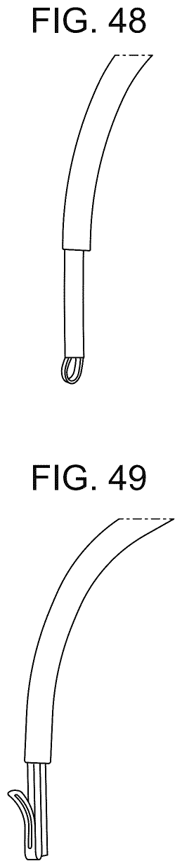

FIG. 49 is an illustration of an anchor in an expanded deployed position.

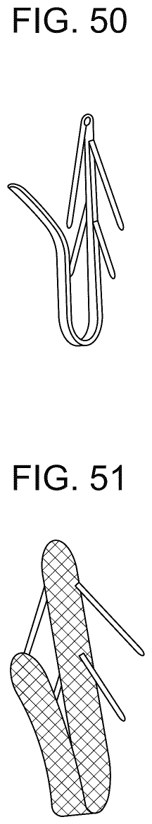

FIG. 50 is an illustration of an embodiment of a bare metal anchor element 110, according to the invention.

FIG. 51 is an illustration of an embodiment of an anchor element having a polyester cover 174 for in-growth along the shank of the anchor, according to the invention.

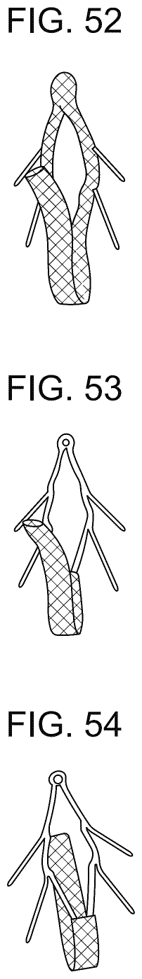

FIG. 52 is an illustration of an embodiment of a split shank 140 anchor element having a polyester cover 174 along the split shank of the anchor, according to the invention.

FIG. 53 is an illustration of an embodiment of a split shank anchor element having a bare metal split shank and a polyester cover 174 on the bend or hook portion of the of the anchor, according to the invention.

FIG. 54 is an illustration of an embodiment of a split shank anchor element having a bare metal split shank and a polyester cover on the bend or hook portion of the of the anchor, according to the invention.

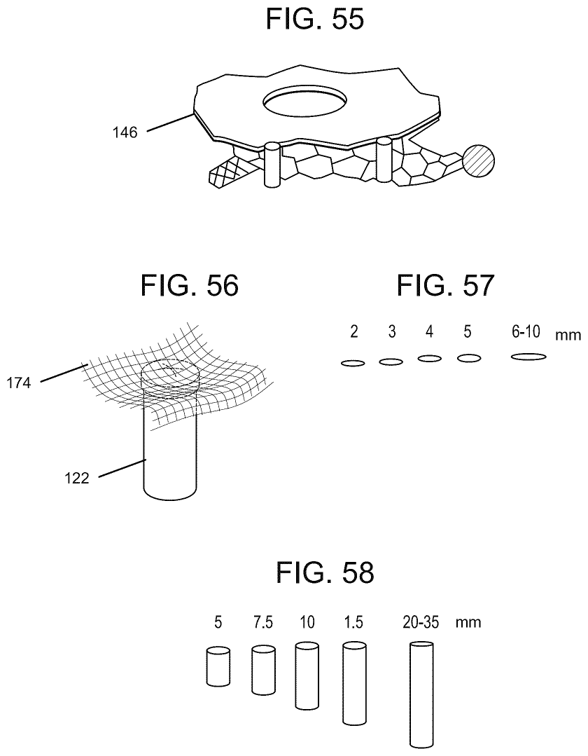

FIG. 55 is an illustration of a side perspective view of an orthogonally deliverable transcatheter heart valve 146 with external anchor channel(s) positioned on the exterior of the valve, and extending from cuff/collar along the outer side surface of the valve body to the subannular/lower side, with distal right ventricular outflow tract (RVOT) tab, and proximal tab also shown, according to the invention.

FIG. 56 is an illustration of a side perspective view of a channel 122 having a mesh or tissue channel cover 174. This allows delivery to effectively puncture the mesh and create a substrate to encourage in-growth.

FIG. 57 is an illustration of varying diameter sizes of the channel(s).

FIG. 58 is an illustration of varying channel length(s) of the channel(s).

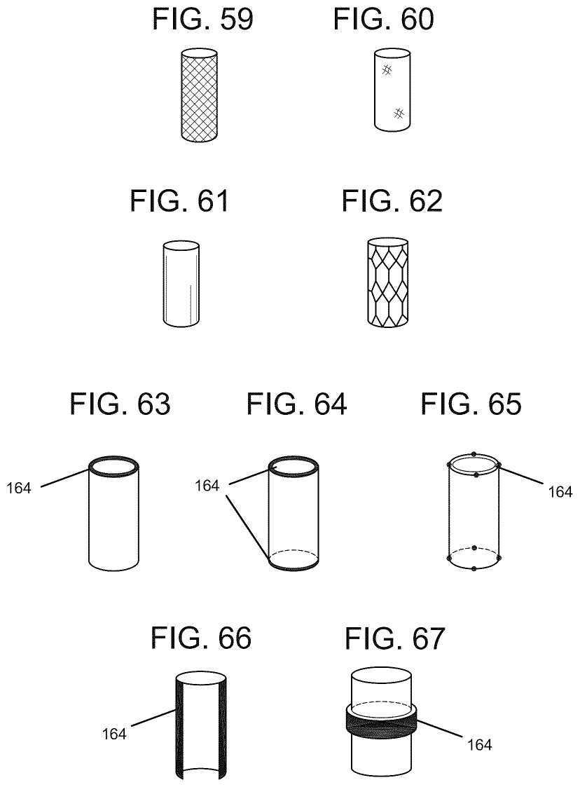

FIG. 59 is an illustration of a side perspective view of a preferred embodiment of a channel and shows braided polyethylene as material for the channel.

FIG. 60 is an illustration of a side perspective view of a preferred embodiment of a channel and shows tissue as material for the channel.

FIG. 61 is an illustration of a side perspective view of a preferred embodiment of a channel and shows expanded polytetrafluoroethylene (ePTFE), as material for the channel.

FIG. 62 is an illustration of a side perspective view of a preferred embodiment of a channel and shows Nitinol® tube or stent, as material for the channel.

FIG. 63 is an illustration of radio-opaque marker 164 locations on a channel, and specifically a top ring location. Material choices for marker 164, according to the imaging modality that it could be used with, e.g. metal marker contrasts well with surrounding valve material under fluoro.

FIG. 64 is an illustration of radio-opaque marker locations on a channel, and specifically top and bottom ring locations.

FIG. 65 is an illustration of radio-opaque marker locations on a channel, and specifically top and bottom alignment dot locations.

FIG. 66 is an illustration of radio-opaque marker locations on a channel, and specifically a side strip(s) location.

FIG. 67 is an illustration of radio-opaque marker locations on a channel, and specifically a mid-band location.

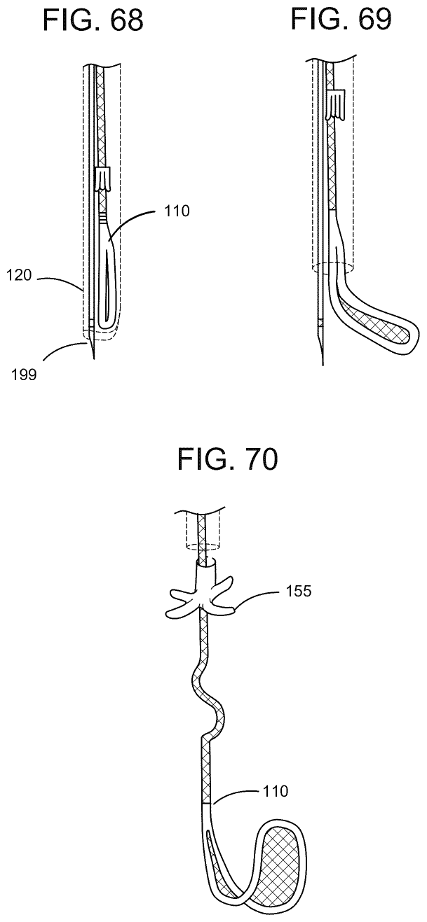

FIG. 68 is an illustration of a side view of a channel-less anchor deployment system with a tunneling catheter 199 having a guide rod, a compressed tether lock, a compressed subannular anchor 110, disposed in a compressed configuration within the catheter 120, according to the invention. This system enables use of the anchor directly through the body of the valve, without requiring a channel. This anchoring system could be used with any valve, not just one that has channels.

FIG. 69 is an illustration of a side view of a channel-less anchor deployment system with a tunneling catheter having a guide rod, a compressed tether lock, a partially expelled, partially uncompressed subannular anchor, according to the invention.

FIG. 70 is an illustration of a side view of a channel-less anchor deployment system with a tunneling catheter having an uncompressed expanded tether lock 155, and an uncompressed subannular anchor 110, according to the invention.

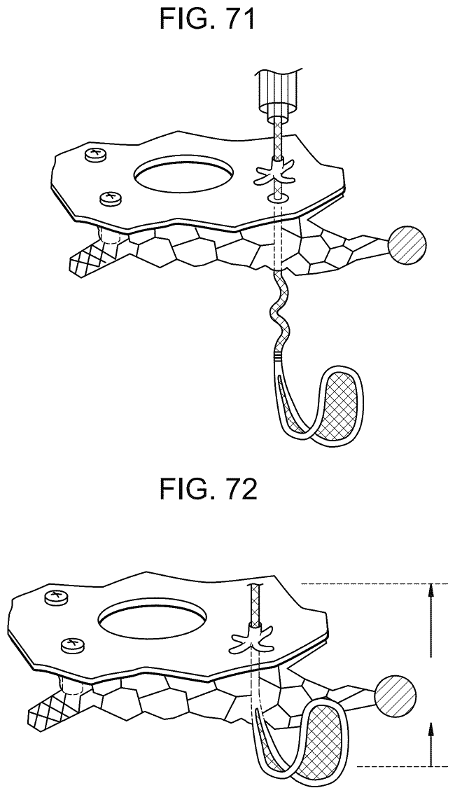

FIG. 71 is an illustration of a side perspective view of an orthogonally deliverable transcatheter heart valve with channel-less pre-determined anchor pathway(s) extending from cuff/collar through the valve body to the subannular/lower side, and distal right ventricular outflow tract (RVOT) tab, a proximal tab according to the invention according to the invention, with anchor delivery catheter, having sub-annular anchor connected by flexible tether to expandable tether lock, prior to cinching the lock down onto the collar surface and the tab against a lower portion of the valve body.

FIG. 72 is an illustration of a side perspective view of an orthogonally deliverable transcatheter heart valve with channel-less pre-determined anchor pathway(s) extending from cuff/collar through the valve body to the subannular/lower side, and distal right ventricular outflow tract (RVOT) tab, a proximal tab according to the invention according to the invention, with anchor delivery catheter, having sub-annular anchor connected by flexible tether to expandable tether lock, after cinching the lock down onto the collar surface and the tab against a lower portion of the valve body.

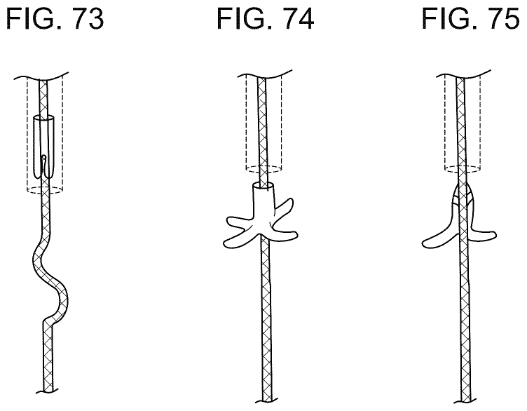

FIG. 73 is an illustration of a side view of a tether lock that is compressed within a delivery sheath (catheter), with the tether lock having a central aperture, with the braided tether threaded thru the central aperture of the tether lock so that the tether lock can slide along the tether.

FIG. 74 is an illustration of a side view of a tether lock that is released by withdrawal of the sheath into an expanded, uncompressed configuration, with the expanded tether lock having a central aperture, and with the braided tether threaded thru the central aperture of the tether lock, and the central aperture having one or more locking teeth to tighten onto the tether and prevent further sliding along the tether. The locking teeth can be a curved, one-way type of pawl or tooth, or they may be piercing teeth that engage the tether when the tether lock is expanded.

FIG. 75 is an illustration of a cross-sectional side view of a tether lock that is released by withdrawal of the sheath into an expanded, uncompressed configuration, with the expanded tether lock having a central aperture, and the central aperture having one or more locking teeth to tighten onto the tether and prevent further sliding along the tether. The locking teeth can be a curved, one-way type of pawl or tooth, or they may be piercing teeth that engage the tether when the tether lock is expanded.

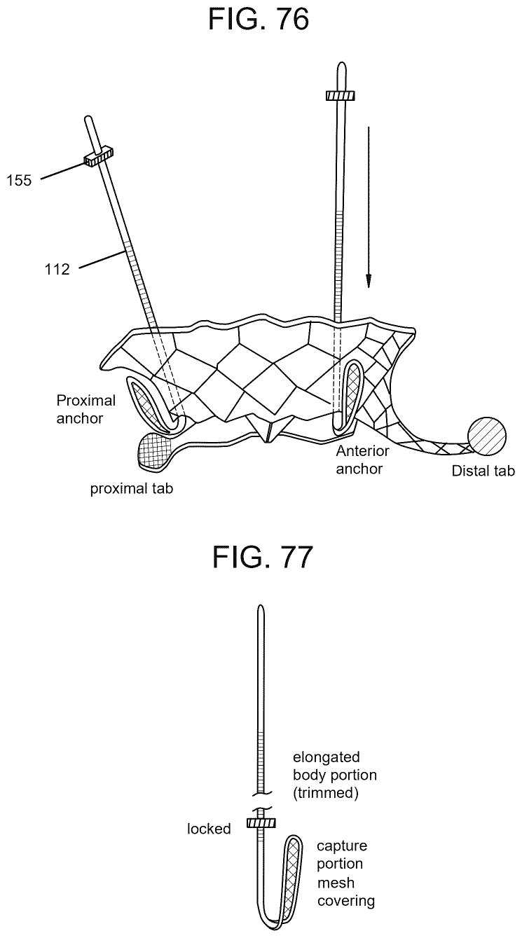

FIG. 76 is an illustration of another preferred embodiment of the invention and shows a compressible valve having a P anchor (near posterior leaflet) and an A anchor (near anterior leaflet), with the tissue anchor loops attached to flexible elongated straps or rods 112 similar to a cable tie, the flexible elongated strap having an attached anchor lock 155 that engages with a section of the strap having teeth, the anchor lock forming an encircling head with a pawl in the head that engages the teeth of the strap, such that when the anchor lock/head slides down the flexible elongated strap, the anchor lock/head is locked into place when the pawl prevents the anchor lock from sliding up the strap.

FIG. 77 is an illustration of a side view of a tissue anchor loop attached to a strap with anchor lock slid into a lower, locked position.

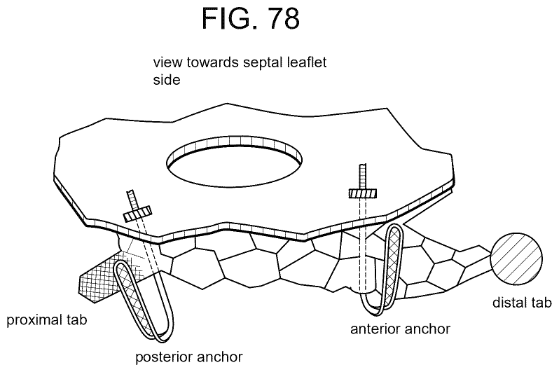

FIG. 78 is an illustration of a side perspective view towards a septal leaflet side of a tricuspid valve replacement with an upper collar attached to a body portion, with an RVOT tab, a proximal tab, a P anchor and an A anchor, with anchor locks slid down into a lower locking position.

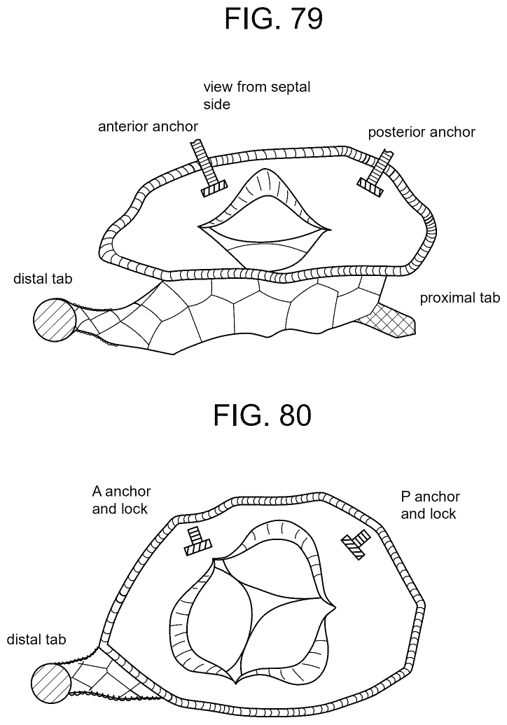

FIG. 79 is an illustration of a side perspective view from a septal leaflet side of a tricuspid valve replacement with an upper collar attached to a body portion, with an RVOT tab, a proximal tab, a P anchor and an A anchor, with anchor locks slid down into a lower locking position.

FIG. 80 is an illustration of a top view of a tricuspid valve replacement with an upper collar attached to a body portion, with an RVOT tab, a proximal tab, a P anchor and an A anchor, with anchor locks slid down into a lower locking position.

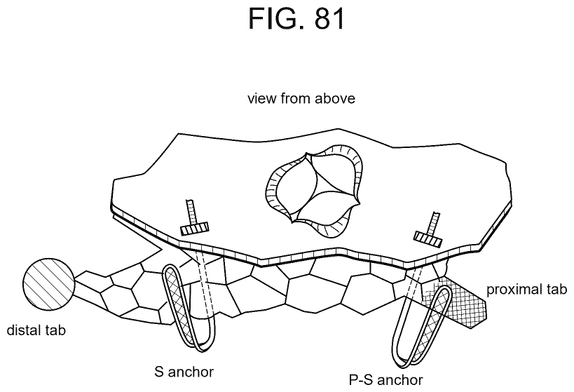

FIG. 81 is an illustration of a side perspective view from above a septal leaflet side of a tricuspid valve replacement with an upper collar attached to a body portion, with an RVOT tab, a proximal tab, central flow control element (tri-leaflet valve), an S anchor (anchor near septal leaflet) and a P-S anchor (anchor near postero-septal leaflet commissure), with anchor locks slid down into a lower locking position.

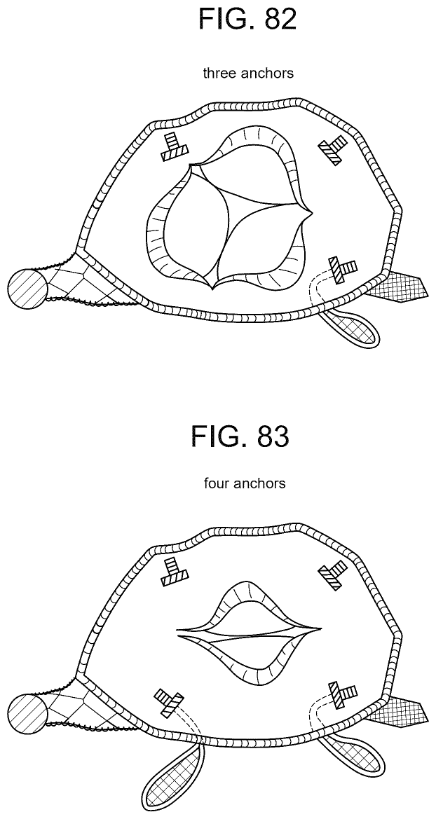

FIG. 82 is an illustration of a top view of a three-anchor tricuspid valve replacement with an upper collar attached to a body portion, with an RVOT tab, a proximal tab, central flow control element (tri-leaflet valve), an S anchor (anchor near septal leaflet), a P anchor (anchor near posterior leaflet), and an A anchor (near anterior leaflet), with anchor locks slid down into a lower locking position.

FIG. 83 is an illustration of a top view of a four-anchor tricuspid valve replacement with an upper collar attached to a body portion, with an RVOT tab, a proximal tab, central flow control element (tri-leaflet valve), an S anchor, a P anchor, a P-S anchor, and an A anchor, with anchor locks slid down into a lower locking position.

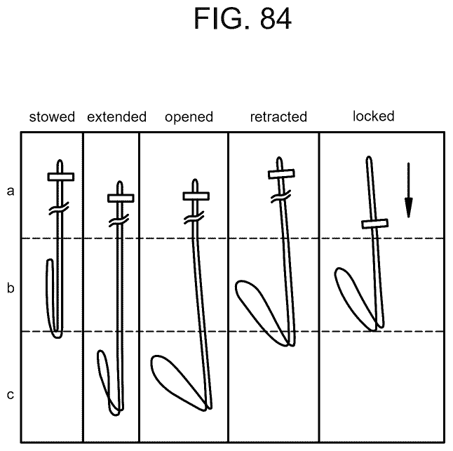

FIG. 84 is an illustration of a side view of a series showing how an anchor can progress from (i) a stowed position within a delivery catheter sheath, (ii) an extended anchor position, (iii) an extended open position, (iv) a retracted position, and (v) a locked position, with an indicator of relative depths A-B-C referring to (A) above the annulus, (B) at or near the annular plane, and (C) below the annulus, according to the invention.

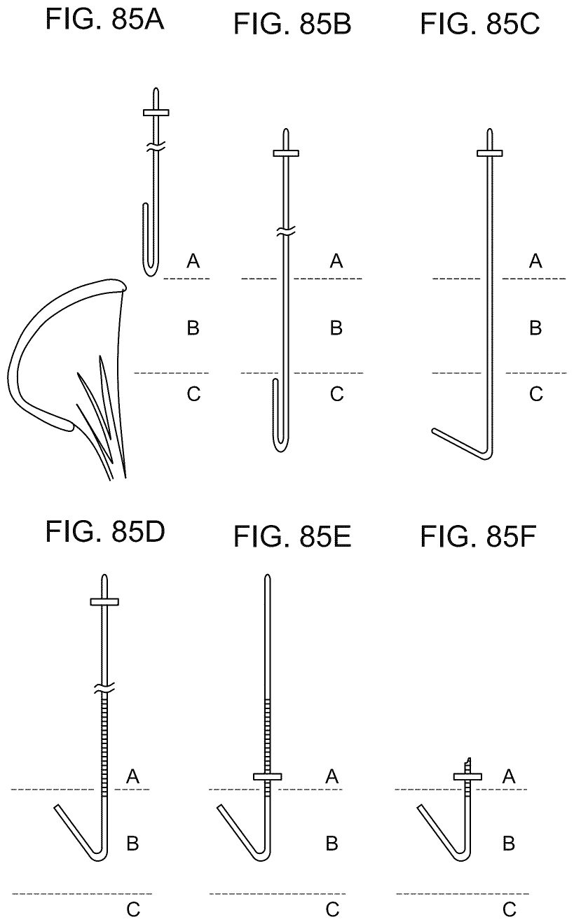

FIGS. 85A-85F are illustrations of side perspective view of the relation of the anchor(s) to native tissue during the anchoring-tissue capture process, with an indicator of relative depths A-B-C referring to (A) above the annulus, (B) at or near the annular plane, and (C) below the annulus, with anchor configurations including (a) compressed shown in FIG. 85A, (b) extended shown in FIG. 85B, (c) opened shown in FIG. 85C, (d) retracted shown in FIG. 85D, (e) locked shown in FIG. 85E, and (f) trimmed shown in FIG. 85F, according to the invention.

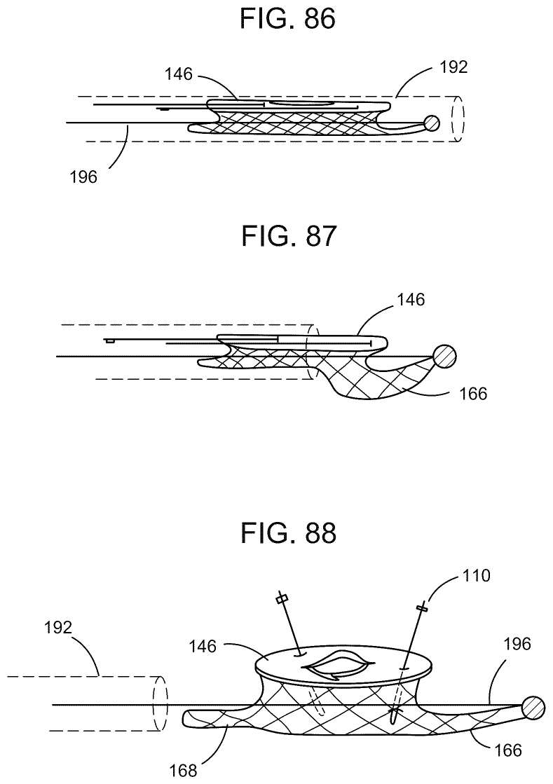

FIG. 86 is an illustration of a side view of an orthogonally compressed valve 146 within a valve delivery catheter 192, with pre-attached folded over elongated straps and anchor locks and with advancement (towing) wire 196 attached to the RVOT tab 166 to pull the valve out of the sheath by applying force to the distal end, according to the invention.

FIG. 87 is an illustration of a side view of a valve 146 partially expelled from the delivery catheter by the advancement (towing) wire, with pre-attached anchor 110 having folded over elongated straps and anchor locks, according to the invention.

FIG. 88 is an illustration of a side view of a fully released valve 146, with pre-attached elongated straps and anchor locks 110 released from the catheter 192 and in ready position for anchor deployment and tissue capture, for the valve 146 having distal tab 166, and proximal tab 168, according to the invention.

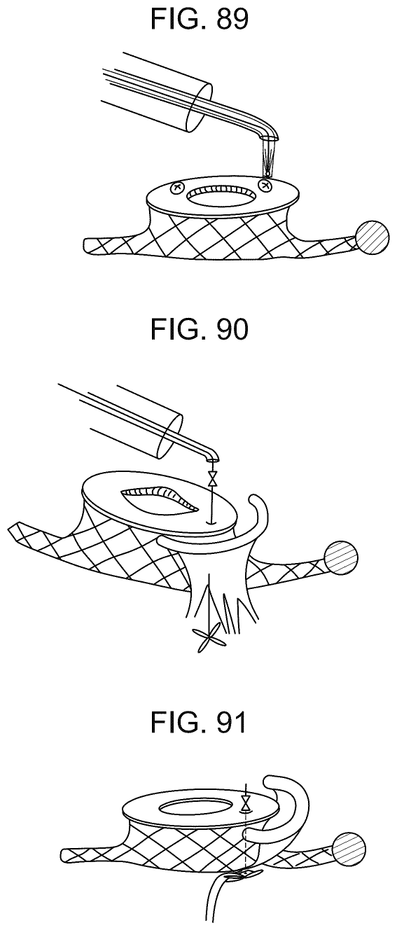

FIG. 89 is an illustration of a side perspective view of an anchor delivery catheter delivering an anchor to a radiomarker spot on the valve collar, according to the invention.

FIG. 90 is an illustration of a side perspective view of an anchor deployed to a sub-annular position, and a tether lock mounted on the tether above the valve collar ready for the tissue cinching step, according to the invention.

FIG. 91 is an illustration of a side perspective view of an anchor deployed to a sub-annular position, retracted to capture the chordae and part of the anterior leaflet, with the tether lock slid down the tether to the valve collar to lock the anchor and cinch the native tissue, according to the invention.

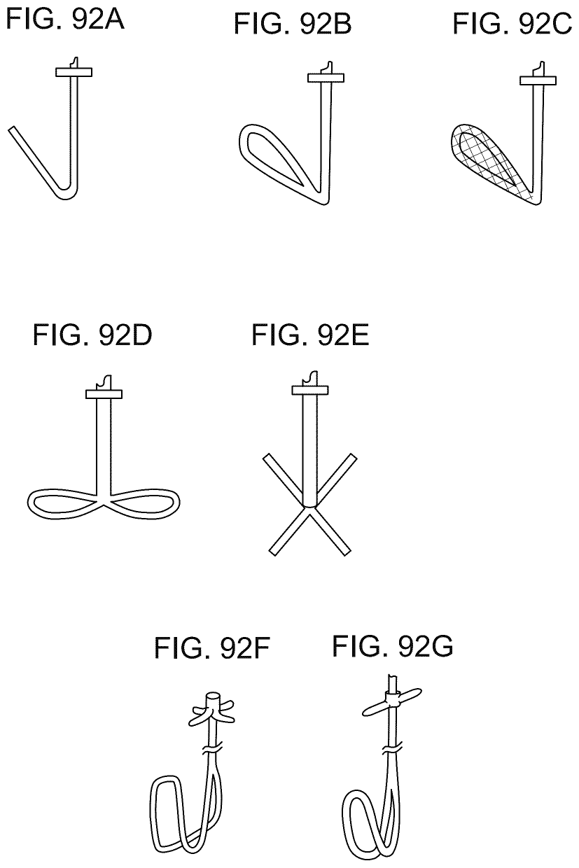

FIGS. 92A-92G are illustrations of a variety of anchor loops and tissue capture structures, according to the present invention.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

Having described embodiments for the invention herein, it is noted that modifications and variations can be made by persons skilled in the art in light of the above teachings. It is therefore to be understood that changes may be made in the particular embodiments of the invention disclosed which are within the scope and spirit of the invention as defined by the appended claims. Having thus described the invention with the details and particularity required by the patent laws, what is claimed and desired protected by Letters Patent is set forth in the appended claims.

What is claimed:

1. An anchoring system for a transcatheter prosthetic heart valve, the anchoring system comprising:
a delivery catheter sized and configured to be at least partially disposed in and at least partially moved through an anchor channel of the transcatheter prosthetic valve;
an anchor hook sized and configured to be at least partially disposed in and at least partially moved through a delivery catheter, the anchor hook having an elongated shaft portion, a plurality of anchoring tabs disposed along the elongated shaft portion, and a hook portion disposed at a distal end of the elongated shaft portion;
an elongated pusher releasably engageable with a proximal end of the elongated shaft portion and configured to extend proximally through the delivery catheter, the elongated pusher configured, in response to a distally directed force, to move the anchor hook in a distal direction to a position in which (i) the anchoring tabs are distal to the delivery catheter and at least partially expanded to engage an interior surface of the anchor channel and limit further movement of the anchor hook in the distal direction, and (ii) the hook portion is distal to the anchoring channel and at least partially expanded to capture native leaflet tissue or native chordae tendineae; and
a tether connected to a tether mount of the hook portion and configured to extend proximally through the delivery catheter, the tether configured to selectively retrieve the anchor hook in response to a proximally directed force that causes the tether to pull the hook portion to a compressed configuration and to withdraw the anchor hook in a proximal direction into the anchor channel.

2. The anchoring system of claim 1, wherein the elongate shaft portion forms a proximal tether loop at a proximal end of the anchor hook.

3. The anchoring system of claim 2, wherein the tether is configured to extend proximally through the proximal tether loop at the proximal end of the anchor hook.

4. The anchoring system of claim 1, wherein the delivery catheter is configured to extend through a valve body portion of the transcatheter prosthetic heart valve via the anchor channel, the anchor channel being integrated into or attached to one of an interior surface or an exterior surface of the valve body portion of the transcatheter prosthetic heart valve.

5. The anchoring system of claim 1, wherein the elongated shaft portion is selected from a single elongated member with the anchoring tabs extending away from the elongated member, or a set of diamond-shaped cells with the anchoring tabs extending from lateral points of the diamond-shaped cells.

6. The anchoring system of claim 1, wherein the hook portion is one of a single diamond-shaped cell or a diamond-shaped cell with one or more anchoring tabs extending from lateral points of the diamond-shaped cell.

7. The anchoring system of claim 1, wherein at least a portion of the anchor hook is configured to be compressed within the anchor channel,
wherein the anchor channel extends through a valve body of the transcatheter prosthetic heart valve vertically or at an angle up to 45 degrees from vertical, and
wherein vertical is parallel to a central atrial-to-ventricle axis of the transcatheter prosthetic heart valve.

8 .The anchoring system of claim 1, wherein the plurality of anchoring tabs includes between 2-6 tabs configured to engage the interior surface of the anchor channel when the anchor hook is distal to the delivery catheter.

9. The anchoring system of claim 1, wherein the anchoring tabs are barbed.

10. The anchoring system of claim 1, wherein the elongated pusher is configured to pull, in response to a proximal force applied on the elongated pusher and after the hook portion transitions to the expanded configuration, the anchor hook proximally into the anchoring channel and causes the hook portion to press the native leaflet tissue or native chordae tendineae against a subannular portion of the transcatheter prosthetic heart valve or a subannular surface of a native annulus of a heart.

11. The anchoring system of claim 10, wherein the anchoring system is further configured such that the proximal force on the elongated pusher pulls the anchor hook proximally into the anchoring channel and causes the tabs or barbs in their expanded configuration to be at least partially compressed and pulled into the anchor channel such that the tabs or barbs press against the interior surface of the anchor channel creating a tension force therebetween operable to limit movement of at least the hook portion in the distal direction.

12. A method for deploying an anchoring system for a transcatheter prosthetic heart valve, the method comprising:
advancing a delivery catheter through a lumen of an anchor channel of the transcatheter prosthetic heart valve to a subannular position;
expelling an anchor hook out of a distal end of the delivery catheter by applying a distal force to an elongated pusher that is disposed within the delivery catheter and engaged with a proximal end of a shaft portion of the anchor hook, the anchor hook having a hook portion on the distal end of the shaft portion, the hook portion being biased from a compressed configuration in which the hook portion is folded towards the shaft portion and an expanded configuration in which the hook portion extends away from the shaft portion, the anchor hook further having anchoring tabs disposed along the shaft portion, the anchoring tabs configured to be in a compressed configuration when in the delivery catheter and configured to transition when advanced beyond the delivery catheter to an expanded configuration in which anchoring tabs extend away from the shaft portion to selectively engage an inner surface of the anchor channel,
wherein the hook portion and the anchoring tabs are in their respective compressed configurations when disposed within the delivery catheter, and the hook portion and the anchoring tabs configured to self-expand to their respective expanded configurations when expelled from the delivery catheter; and
pulling the shaft portion of the anchor hook into the anchor channel by pulling the elongated pusher in a proximal direction,
wherein pulling the shaft portion retracts the hook portion in a proximal direction and captures native leaflet tissue or native chordae tendineae within or on the hook portion of the anchor hook, and the retracted hook portion presses the native leaflet tissue or native chordae tendineae against a subannular portion of the prosthetic transcatheter heart valve or a subannular surface of a native annulus, and
wherein pulling the shaft portion of the anchor hook into the anchor channel draws the anchoring tabs in the expanded configuration into the anchor channel and create a tensioning force with the anchoring tabs pressing against an interior surface of the anchor channel.

13. The method of claim 12, wherein the anchor channel is a mesh or braid and the tabs are configured to engage the interior surface of the anchor channel.

14. The method of claim 12, further comprising:
retrieving the anchor hook, wherein the delivery catheter slides over the shaft portion while the tabs or barbs are engaging the anchor channel, and flattens the tabs or barbs back towards or against the shaft portion, thus disengaging the tabs or barbs from the anchor channel,
wherein the delivery catheter is advanced distally to a subannular/ventricular position to release the hook portion from the captured native tissue,
wherein a tether that is attached to the anchor hook is pulled proximally and folds the hook portion against the shaft portion,
wherein the anchor hook is then pulled into the delivery catheter, and
wherein the delivery catheter is withdrawn and/or the anchor hook is redeployed.

15. A method of using the anchoring system for orthogonal delivery of an expandable transcatheter prosthetic heart valve to a desired location in a body, the method comprising:
advancing a delivery catheter to the desired location in the body and delivering the expandable prosthetic heart valve with the anchoring system of claim 1 to the desired location in the body by releasing the valve from the delivery catheter, and
anchoring the anchor hook attached to the valve into native tissue,
wherein the valve comprises an annular support frame having a flow control component mounted within the annular support frame and configured to permit blood flow in a first direction through an inflow end of the valve and block blood flow in a second direction, opposite the first direction, through an outflow end of the valve, the anchor hook attached to the annular support frame, a distal anchoring tab mounted on a distal side of the annular support frame, and a proximal anchoring tab mounted on a proximal side of the annular support frame,
wherein the valve is compressible to a compressed configuration for orthogonal delivery into the body using a delivery catheter, the compressed configuration having a long-axis oriented at an intersecting angle of between 45-135 degrees to the first direction, and expandable to an expanded configuration having a long-axis oriented at an intersecting angle of between 45-135 degrees to the first direction, and
wherein the long-axis of the compressed configuration of the valve is substantially parallel to a length-wise cylindrical axis of the delivery catheter.

16. The method of claim 15, wherein releasing the valve from the delivery catheter includes:
pulling the valve out of the delivery catheter using an elongated pushing rod that is releasably connected to the distal side of the valve, wherein advancing the pushing rod away from the delivery catheter pulls the compressed valve out of the delivery catheter, or
pushing the valve out of the delivery catheter using the elongated pushing rod that is releasably connected to the proximal side of the valve, wherein advancing the elongated pushing rod out of from the delivery catheter pushes the compressed valve out of the delivery catheter.

17. The method of claim 15, wherein the anchoring of the anchor hook into native tissue includes expelling the anchor hook from the anchor channel, extending the hook portion of the anchor hook, capturing native tissue, and pulling the tether to compress the anchor hook.

18. The method of claim 15, further comprising:
positioning the distal anchoring tab of the valve into a right ventricular outflow tract of a right ventricle.

19. The method of claim 15, further comprising:
positioning the distal anchoring tab of the valve into a right ventricular outflow tract of a right ventricle; and
positioning an upper distal anchoring tab into a supra-annular position, and the upper distal anchoring tab providing a supra-annular downward force in the direction of the ventricle and distal anchoring tab providing a sub-annular upward force in the direction of an atrium.

20. The method of claim 15, further comprising:
rotating the valve using a steerable catheter along an axis parallel to a plane of a native valve annulus.
seating the at least one proximal anchoring tab at a second desired anchoring area; and
capturing native tissue via the anchor hook.
